SEQUENCE LISTING (15 sequences)

SEQ ID NO: 1 - DNA, 7010 bp, Tilapia nilotica
  misc_feature (1538)..(1538): n is a, c, g, or t
tgatccggagaccccaccgcccaacatgaagcctctcctcctagtgacgtctcttggtgatccggagaccccaccgcccaacatgaagcctctcctcctagtggcgctgcttggactgctcggtaaaagcttttcagccccgcactacgaagtaggcaaagagttcgctgcacccggctctcagaaggtgaaatggtgcgtcaagtcagaccaagaattgcgtaaatgttcagatctcgccgctgcgattccttgcgtcatctgtgtgaaaaaagaaaacaccctagagtgcattgttgccattaaggtgagcgctttttacgtccagagcaccaaattaaccacaaatgctcaataacagactttgacctgggcaattgttataaacgtaacawttctgataarcgtataartgtaactgaagtgcttcacaggtgctctgcttattctgaaatgttacttaagtaaaaataagtgaatgtaatgacttcattttattgttttttttcttcactttcttcgagtaggttcatactcgaactactactaaaacgtatctaaagtaaagataaattaactgtatctgctgtggagtgttctagaaaaattgagtagagttaaaaatattaaaaaaaattaaatccgaaccaaatgcttttttaactatttcaattgcatctaaatattcaatttaatttggttacataaatacaatttaaatgttacatttaatggaagcttgtcaaataaaatgtatttgattggcatggattcagtttataccaagcacattttccttgattattatttagctcattaaagaaacatttgattttatatatttcagctttactcactttactcagaatkattacatcatgtttgstgaggatgaataattttttttaaacccttttaagyatttaatgtggggaaaaaactaccaatgccttcaactcattcagtgaaattctctgtaaaaatgacttgacctcagtcatacaacaaatactctcgggcaggctggtgaagcagatgccattactgtggacggtggggacatttacactgctggcttgaacaactacgacctccatcccatcattgctgaggactacggcaccggtatgtccactcatcagctgtcattagtagcctaatgaaataagctcaagcaagaacatgtttaccgttttatgtgaatgtgatttagctgtttcaataatatgtatccattttcatcattcattcagcttcggacacctgctactacgctgttgctgtggcaaagaagaatactggatttggcttcagagatctcagggggaagaaatcatgccacactggtttggggaaatctgcaggctggaacattcccataggatctctggtgtccatgaatgttattgagtggggtggcattgaagacaaacctttggaggaaggtgagggacttttttttctggtgagctcataacatttgacagctttagtgcatgtatggtcgaaggacgataagatcagacctttttatgagnaactggacgcctgagaaacaattaaacacacttatttgctttactgacgagaatttattgatgggatcaaaactacaggaaaagtagaaacagacagggaaggggctacctagcttcttttttattagtcatttatttaaccaggtaaaaaaatcctcattgagattaaaaatctctttccaagagagaccttgcttctgcccagggattataaaatcctgtgcctgtcagcacttcttatcacttataacacttccttttgttaaatataattaactgaagttcaaaaactagggcgttttagactagttaggccctgttagacagattgaacaaatcaatatccatccatcttcttctaaccaaccaccggacacgacaggggctggaggcttgcctctcagtgctgagaggggggaaaggtcgccagccatgtactcacattcgtacatatggtcagtttagaactgacaattaacctaacatgaaagaaactggtgtatccagtatcccatggatgcaaactctgcacacacaggataatatgaaacttctaaatgttaaaaatgcatatgggaactttaatatttgtctttaaaggcagacagtggtgacactctggctcttctgtcagtgttttggttttcttttctaaattatgactgtggccttgtttgattttgttatgcagcggtgagcaccttcttccatgccagctgtgcccctggggcagcaagaggcagcaagctgtgtgaactgtgcaagggagactgctccaggtcccagagggagccttactatgactacaatggagcctttcagtcagtgtccactttttaatgcttctcattagtgaacataaacttctacacatttagccgaaccttttatctgtaataatatgacttttgtaacaggtgtttggccgagggagctggagatgtggcttttgtgaagcatctcactgtgccaggtcagtatcacaacaacctttatccattcgaacatataaagaagcactgcatcatagcattgtcaggttttattcatcaaaaacgagcctcaccgagattaaaaatttcattttgaaaagcgactgggacaagaagggacctgatgaacatgcatgacatacaaatacaactgttgcacactgcacaagtcaaggacaagtcaagcagaatttcaaagcaaatttcagagcaatcacattgaccaagagtgtcattttctcaaatgattaaatggtcttaaacaccctgaaacttctacaaatatgggaaagaataactgtgaaaaacactgatgtaaaacctgtgtcatgtcacacttccagaatcggataagccaatgtacgagctactgtgcaaggacaacaccagagcacctattgacaactataacacctgcttcctggccagagtgccagctgacgctgttattactcgcaaggatccacagctggccgactttatctgggagacccttgaccgagttcagactgaccacgtaggaagtccactgaataacatctcatttcaacgtattctgattctttgtgtttcagcagcctcattgctttcctaatgaatcatctcacttgctgccttttcagagctttaacctcttctcatctgaagcctacgcgcccgccaagaacctgatgttcaaagattcaacagtgaggctcttgagggtgcccccaaacacagactccttcctgtatctgggtgctaactacatgagcatcgtccattcccttaaaaaaggtaaagaacacaagcaagacagacagaaacaattgttagtcaggtgtcggtctggcttatgtctaagtctttttcccgtctctagagcaggcatcagacgttgcatcccctgccattaggtggtgtgctgtgggccatgctgagactgctaagtgtgacacgtggagcatcaacagtgtgtctgatgacaccgcctccattgaatgccagagcgcccctacagttgaagattgcctgaagaagattatggtaaactcagtaggaaatatcgaaatctgttactttattattttttgactccactctgtgacgttcatctctgctttgaatcgccccgcagcgtaaagaggctgacgcagtcgccgtggatggaggtcaggtgtttacggctggaaagtgtggcctggttcctgtcatggtggagcagtatgaccaaggtagataacaagaagaagaaataacacacacacaatcctgtgtcgtatttcaccttttctatatgagtaatatttgttcattttgtcttctctcgcagagatgtgcggcaactctaatggtagtaaactttcatctttttcctcctctttttatgatctggacaaattttttaaaaagcctcttgaagtgcatcattttaattgtaacacagcttcgatgacctgttgtgctgatgacaagatttgtattttatatttgtatccttggactctaccactctactactacaactacaactataatttatatgaacaaccagttcaaggaatccttatttattcattagtaaccccttagttcatcctctgaaacaaacctctgcggaagctttgaacaacaggtggatggagcttgtcttcattatatattaaggatatagtctctttatggctttacaaacaaccaagatttttaaaaactacaaaaaaattccaaattgagcatttacataaatctggagacctagcatttagctcattggcattgcttgtacattgtctaaatttttagtggctccggttttgtaagtatccacgattttaatgatagattacctttaaaaagtggattaattttaagcagccgtaaacagagtagatttatatgaacgtctctctccagttgaatcatctgaaatgtctacttcccgtatccactctcagccccagcctcctcatactatgctgttgctgtggtaaagaagggttcaggggtgacctgggagaatctgaagggcaagaagtcttgccacacaggcatcggcagaactgctggctggaacatccccatgggtctaatctacaacagagaacatgactgtgacttcagtgagtgactgcgagcttcttccttcttttaccaacagaaagaaatttaaagccacccacagaaatcatacaactcccctttttctaaaaaaaaagtttgtttagatgactcatgtgattaatctatatttaaacaaaagccacaatcttccattaaccttgacaaaagagaggtttgctgattgttgagctctttctgcatctcttcctctcctcagctaagttcttcagtagcggctgtgcccccggagcagagcccacctctccgttctgttctctgtgtgtcggcagcggtaaagctgtgggagacgaggccaaatgcaaagccagcgctgatgagaagtactacggttatgctggagcctttaggtacagcaagamacagaatggcatcgcatacaagtttgtgtataaggagaaagttagcaaagagacaggaagcactggaaaacaacagaaccagctcttctsaatsttggcagctactgttatgttttttttcttatatatttcatttgtaaacagccaagaatgtgtcaagagctgaagcatgttttcctcctaaaaccaggactttttaatctttttttaaattgttgtgaactttagcaatgatgaggtgtaatttttaattggttagccatttacccccaacttcactctttgtctccaaagtcatgatgtatggcatatcattacaggtgtctggttgagggtggtggtgatgttgccttcgttaaacacacaactgtgacagaaaacagcgatggtaagaggactgcactgatctgcactacctgctccgatgacattacaacagcttctaaagtttactccacctctctttaccgaaggtaacggtccagactgggctcgtaatctgagatcgtctgattaccagctcatctgccctgggaagggaccagtggagatcagtgattatgccacttgtaacttggctcaggcgcctgcacacgctgtggtgactcgcccagaaagccacagtaaggtggtccgcatccttcaggaacagcaggtttgtgataaagtctaagagagcyacttgaaatgaaataagagsttaaaataaaattgttgttgtttcaaagcactgaattcctagtgcaaaaatctkacatcrmggttcaacttttctagctgacacagatgctgagtctgaaatagagaagcatcctgtcattacagaagctgcatacagtttgtttggcattctgctttgagcacaacaatgaatttttaacagattttccaaatagttgccaattaactctgacaactttaacattgtccctttgtaggggctacatctaagactctacaggcctcagttaacatgttaaatgttaaagcttatgacagcagaattagaaaaagactgagcaaatgtagcttgtttggaaagaaaaagccgcttctctctacagagaacatggcagcacagcttttgtttgcagagttgcatctgaacaaaccaagagacttctggaacaatgttcgtctggccttaatgtacaggaccacgtttgttgaaaatcaaacatagcatattcgcacaaacacctcatagaaactggtggtggaggggtgttgatttgggcttcttttacagcaacgtggcacaaacatttcaagttactgctcccaaagttgattctacttgctactgaatcatggagtgtacttagtttgtcacaactgcatagagtcctgtgaaaactttctttttgatatgactgtacttgcacttgatctcaagtggggggaccttggcaattcaaaaaccaatagtgacttctgtttttctacattctgccggtttcaatgagtaaagtaaccagtgtaatagaaatctaaaaattaaataactgcttcaaacctaaattttgttcataggccagatttggaaacgctgggactgatccttcattcagaatgttccagtcagagaatggaaagaaccttctcttccaagactccactaagtgtctccaggaggtaccagaggaaagaacctatgagcagtttttgggagaagcttacatggatgccatgaaggcactgagacattgcagcgatactgcttcaggtttgtactactgcagaaattacttagcatttaaatgataattttataccatcttgtaccagataaaataacattcatgctcgatattctccaattgtttttgtttgcctctcttcccaacagatctggaaaagtcttgcacattccatgcctgccagcaaggctaatggagatgtagacaacacagtgattcagccatcttacccacttacttcctgcacttacccaaacatagtgtttctcctcttctgatcttactatagactataga

SEQ ID NO: 2 - protein, 694 aa, Tilapia nilotica
MKPLLLVALLGLLGKEFAAPGSQKVKWCVKSDQELRKCSDLAAAIPCVICVKKENTLECIVAIKAGEADAITVDGGDIYTAGLNNYDLHPIIAEDYGTASDTCYYAVAVAKKNTGFGFRDLRGKKSCHTGLGKSAGWNIPIGSLVSMNVIEWGGIEDKPLEEAVSTFFHASCAPGAARGSKLCELCKGDCSRSQREPYYDYNGAFQCLAEGAGDVAFVKHLTVPESDKPMYELLCKDNTRAPIDNYNTCFLARVPADAVITRKDPQLADFIWETLDRVQTDHSFNLFSSEAYAPAKNLMFKDSTVRLLRVPPNTDSFLYLGANYMSIVHSLKKEQASDVASPAIRWCAVGHAETAKCDTWSINSVSDDTASIECQSAPTVEDCLKKIMRKEADAVAVDGGQVFTAGKCGLVPVMVEQYDQEMCGNSNAPASSYYAVAVVKKGSGVTWENLKGKKSCHTGIGRTAGWNIPMGLIYNREHDCDFTKFFSSGCAPGAEPTSPFCSLCVGSGKAVGDEAKCKASADEKYYGYAGAFRCLVEGGGDVAFVKHTTVTENSDGNGPDWARNLRSSDYQLICPGKGPVEISDYATCNLAQAPAHAVVTRPESHSKVVRILQEQQARFGNAGTDPSFRMFQSENGKNLLFQDSTKCLQEVPEERTYEQFLGEAYMDAMKALRHCSDTASDLEKSCTFHACQQG

SEQ ID NO: 3 - DNA, 543 bp, Tilapia nilotica
tccgctattcaaagtgagtcattatctggaacaacactaagctgatatctttaactcttttacggtgttttaaaaaagcgcatttgagtacttacatttccatcacagctgggacatgatgttgctgagtagactgtgattagcctcttgttgcagaaaacaatgacgttagcttaattttattttactgcatcgcgtttgattgggaatatgatcatttctgtttatttaatgttaatctgtttaatatcttgaaataagcactaatgtatctgatattaattttagttgtttaacaaagcagtagtatgggaatatagtacagtactgagcgaaagtctcaagccattcctcatatacatacatacatacgtgtgtgtgtgtgtgtgtgtgcaagcattcatggaaataaagtatataccattcccattgtctttagagtcactgtcctggttgaaaatgaagccgatgccaatggaatgctttccaggtggtattgcatggtagatcaatggatcagtcccttcaccactggctgaaatg

SEQ ID NO: 4 - DNA, 199 bp, Tilapia nilotica
tagagcgagttaattagaagcgcagttacacatgtggctgttattatcgtaggtgctaattagcagcatttgtcagatgtaaaaataaaaaaaattaaaaaggggggtggggatggcaatcaggaagtgtgtgtgtgtgtgtgtgtgtgtgtgtgcgcgcacgcttaaaagcacctcacgtaattaactgctccagaga

SEQ ID NO: 5 - DNA, 20 bp, Tilapia nilotica
ttttactgcatcgcgtttga

SEQ ID NO: 6 - DNA, 20 bp, Tilapia nilotica
atcggcttcattttcaacca

SEQ ID NO: 7 - DNA, 22 bp, Tilapia nilotica
gcgagttaattagaagcgcagt

SEQ ID NO: 8 - DNA, 22 bp, Tilapia nilotica
tggagcagttaattacgtgagg

SEQ ID NO: 9 - protein, 693 aa, Tilapia nilotica
MKPLLLVALLGLLGKEFAAPGSQKVWCVKSDQELRKCSDLAAAIPCVICVKKENTLECIVAIKAGEADAITVDGGDIYTAGLNNYDLHPIIAEDYGTASDTCYYAVAVAKKNTGFGFRDLRGKKSCHTGLGKSAGWNIPIGSLVSMNVIEWGGIEDKPLEEAVSTFFHASCAPGAARGSKLCELCKGDCSRSQREPYYDYNGAFQCLAEGAGDVAFVKHLTVPESDKPMYELLCKDNTRAPIDNYNTCFLARVPADAVITRKDPQLADFIWETLDRVQTDHSFNLFSSEAYAPAKNLMFKDSTVRLLRVPPNTDSFLYLGANYMSIVHSLKKEQASDVASPAIRWCAVGHAETAKCDTWSINSVSDDTASIECQSAPTVEDCLKKIMRKEADAVAVDGGQVFTAGKCGLVPVMVEQYDQEMCGNSNAPASSYYAVAVVKKGSGVTWENLKGKKSCHTGIGRTAGWNIPMGLIYNREHDCDFTKFFSSGCAPGAEPTSPFCSLCVGSGKAVGDEAKCKASADEKYYGYAGAFRCLVEGGGDVAFVKHTTVTENSDGNGPDWARNLRSSDYQLICPGKGPVEISDYATCNLAQAPAHAVVTRPESHSKVVRILQEQQARFGNAGTDPSFRMFQSENGKNLLFQDSTKCLQEVPEERTYEQFLGEAYMDAMKALRHCSDTASDLEKSCTFHACQQG

SEQ ID NO: 10 - protein, 689 aa, Acanthopagrus schlegeli
MKTLLLVALLGCLAAVFAAPEKVWCVKSDQELRKCNDLMAKAPVFSCVKRDNTIDCIIAIKAGEADAITLDGGDIYVAGLNNYDLHPIIAEDYGTSSETCYYAVAVVKKGSGFGIRDLRGKKSCHTGLGKSAGWNIPVGTLLSMGLIEWTGAEDSPVEEAVSNFFSASCAPGAQRGSKLCELCRGDCSRSHSEPYYDYGGAFQCLADDAGEVAFVKHLTVPAAEKHKYELLRKDNTRAPIDSYSTCHLAKVPAHAVVTRKDPQLAELIWTSLNSVQEFNLFSSEAYSPAKNLMFKGSTVRLVKLPPNTDSFLYLGAEYMRIVRSLKKEPIAATSSSAIRWCAVGATETQKCDAWSINSMTENGAIMGCPNAATVDGCLKKIMRGEADAMAVDGGEAYTAGKCGLVPVMVEQYDQAQCGTNGVTASSYYAVAVVKKGSGVTWENLKGKRSCHTGIGRTAGWNVPMGLIHKQTDDCDFSKFFSEGCAPGADPSSVFCNKCAGSGRGVEDESKCKASSVVQYYGYAGAFRCLVEGAGDVAFIKHTIVDENSNGNGPAWASGVNKDDYELICPNKSPVPVTDYLSCHLARVPAHAVVTRPESRGKVVDFLKDQQSKFGIGGSDPSFNLFQSDLGRNLLFKDSTKCPQEIPAERSYEQFLGAEYMNAMASLRKCTDSTSDLEKSCTFHSCQQKN

SEQ ID NO: 11 - protein, 690 aa, Salmo trutta
MKLLLLSALLGCLATAYAAPAEGRIVRWCVKSEQELRKCHDLAAKVAQFSCLRKDGSFECIQAIKGGEADAITLDGGDIYTAGLTNYGLQPIIAEDYGEDSDTCYYAVAVAKKGTAFGFNTLRGKKSCHTGLGKSAGWNIPIGTLVTENQIQWGGIEDRPVESAVSDFFNASCAPGATKGTKLCQLCKGDCSRSHKEPYYDYAGAFQCLKDGAGDVAFIKPLAVPAAEKASYELLCKDGTRAPIDSYKTCHLARVPAHAVVSRKDPELADRIYNKLVAVKDLNLFSSDGYAAKNLMFKDSAQKLVQLPNTTDSFLYLGAEYMSTIRSLKKSQATGATSRAIKWCAVGHAKKDKCDTWTINSFADGESKIECQNAPTVEECIKKIMRKEADAITVDGGEVYTAGKCGLVPVMVEQYDADLCSAPGASSYYAVAVAKKGSGLTWKTLKGKRSCHTGLGRTAGWNIPMGLIHKETEDCDFTKYFSKGCAPGSEVGSTFCAQCKGSGKPVGDEDMCKARSEEQYYGYTGAFRCLVEGAGDVAFIKHTIVPESTDGNGPVWAKDLKSSDFELLCQDGGTQPVTKFRECHLANVPAHAVITRPETRGDVVSILLEQQAKFGSSGSDSSFKMFQSSVEKNLLFKDSTKCLQEIPKGTKYQDFLGEEYMIAMQSLRKCSDSTSDLEKACTFHSCQQKE

SEQ ID NO: 12 - protein, 688 aa, Oryzias latipes
  misc_feature (486)..(486): Xaa can be any naturally occurring amino acid
MKPLLLLTLLGCLAAALAVPQKVWCVKSDQEFRKCSDLAAASPAFSCVKKESTLDCIIAIKAGEADAITVDGGDVYTAGLNNYDLHPIIAEDYGTSSETCYYAVAVAKKGTTFGIRDLRGKKSCHTGLGKSAGWNIPIGTLVSMDIIQWAGVEDKPVEEEVSTFFQASCVPGATRGSKLCELCKGDCSRSQKEPYYDYNGAFNCLAEGAGDVAFVKHLTVPDQEKSKYELLCRDNTRAPIDDYKKCNLARVPAHAIVTHKDPQLAELIWTSLNSVQNFNLFSSEAYAPSKNLMFKDSTQRLVKLPQNTDSFLYLGAQYMSIVRSLKKEQSVGTNSNAIKWCAVGHAETAKCDTWSINSVTDDTAAIECQNAPSVEECLKKIMRKEADAMAVDGGEVYTAGKCGLVPAMVEQYDAELCSSSGGQASSYYAVAVVKKDSGVTWENLKGKKSCHTGIGRTAGWNIPMGRIYDQTKDCDFTKFFPSGCAXGAEASSSLCTLCKGSGKAVGDEAKCKARPEEQYYGYAGAFRCLAEGAGDVAFIKHTIVGENTDGNGPDWARSLKSDDYQLICPGKGPVPISEYASCNLAVVPAHAVVTRPESRSDVVRVLQVQQTFFGASGSDPSFKLFQSQNGNNLLFKDSTKCLQEVPAGTSYDQFLGSGYMEAMTSLRKCSDTASDLEKSCTFHSCQQT

SEQ ID NO: 13 - protein, 689 aa, Pagrus major
MKTLLLVALLGCLAAVFAAPEKVWCVKSDQELSKCTDLMAKAPAFSCVKRQNTIDCIIAIKAGEADAITLDGGDIYTAGLNNYDLHPIIAEDYGTSSETCYYAVAVVKKTSTFGIRELRGKKSCHTGLGKSAGWNIPVGTLLSMGLIQWSGAEDSPVEQAVSNFFSASCAPGATSDSKLCQLCRGDCSRSHSEPYYDYGGAFQCLADDAGDVAFVKHLTVPESEKHKYELLCKDNTRAPIDSYSTCHLAKVPAHAVVTRKDPQLAELIWTSLNSVQEFNLFSSEAYSPAKNLMFKDSTVRLVKLPPNTDSFLYLGAEYMSTIRSLKKEQITAATSSAIRWCAVGQAETHKCDAWSINSMTDSGTDIECQNAATVEECLKKIMRKEADAMAVDGGQVYTAGKCGLIPAMVEQYDQAQCSTTGGTASSYYAVAVVKKGSGVTWESLRGKRSCDTGVGRTAGWNVPMGLIHKQTGDCDFSKFFSEGCAPGADPTSVFCNKCAGSGKAVGDESKCKANANEQYYGYAGAFRCLVEGAGDVALIKHTIVDENSNGNGPAWASGVNKDDYELICPNKSPVPVSDYLSCNLALVPAHAVVTRPESHGDVVRILQDQQTKFGTSGSDPTFKLFQSDSGKNLLFKDSTKCLQEISAGTTFEQFLGQEYMGAITSLRQCTDSTPDLEKSCTFHSCQQKN

SEQ ID NO: 14 - DNA, 7010 bp, Tilapia nilotica
  misc_feature (1538)..(1538): n is a, c, g, or t
tgatccggagaccccaccgcccaacatgaagcctctcctcctagtgacgtctcttggtgatccggagaccccaccgcccaacatgaagcctctcctcctagtggcgctgcttggactgctcggtaaaagcttttcagccccgcactacgaagtaggcaaagagttcgctgcacccggctctcagaaggtgaaatggtgcgtcaagtcagaccaagaattgcgtaaatgttcagatctcgccgctgcgattccttgcgtcatctgtgtgaaaaaagaaaacaccctagagtgcattgttgccattaaggtgagcgctttttacgtccagagcaccaaattaaccacaaatgctcaataacagactttgacctgggcaattgttataaacgtaacawttctgataarcgtataartgtaactgaagtgcttcacaggtgctctgcttattctgaaatgttacttaagtaaaaataagtgaatgtaatgacttcattttattgttttttttcttcactttcttcgagtaggttcatactcgaactactactaaaacgtatctaaagtaaagataaattaactgtatctgctgtggagtgttctagaaaaattgagtagagttaaaaatattaaaaaaaattaaatccgaaccaaatgcttttttaactatttcaattgcatctaaatattcaatttaatttggttacataaatacaatttaaatgttacatttaatggaagcttgtcaaataaaatgtatttgattggcatggattcagtttataccaagcacattttccttgattattatttagctcattaaagaaacatttgattttatatatttcagctttactcactttactcagaatkattacatcatgtttgstgaggatgaataattttttttaaacccttttaagyatttaatgtggggaaaaaactaccaatgccttcaactcattcagtgaaattctctgtaaaaatgacttgacctcagtcatacaacaaatactctcgggcaggctggtgaagcagatgccattactgtggacggtggggacatttacactgctggcttgaacaactacgacctccatcccatcattgctgaggactacggcaccggtatgtccactcatcagctgtcattagtagcctaatgaaataagctcaagcaagaacatgtttaccgttttatgtgaatgtgatttagctgtttcaataatatgtatccattttcatcattcattcagcttcggacacctgctactacgctgttgctgtggcaaagaagaatactggatttggcttcagagatctcagggggaagaaatcatgccacactggtttggggaaatctgcaggctggaacattcccataggatctctggtgtccatgaatgttattgagtggggtggcattgaagacaaacctttggaggaaggtgagggacttttttttctggtgagctcataacatttgacagctttagtgcatgtatggtcgaaggacgataagatcagacctttttatgagnaactggacgcctgagaaacaattaaacacacttatttgctttactgacgagaatttattgatgggatcaaaactacaggaaaagtagaaacagacagggaaggggctacctagcttcttttttattagtcatttatttaaccaggtaaaaaaatcctcattgagattaaaaatctctttccaagagagaccttgcttctgcccagggattataaaatcctgtgcctgtcagcacttcttatcacttataacacttccttttgttaaatataattaactgaagttcaaaaactagggcgttttagactagttaggccctgttagacagattgaacaaatcaatatccatccatcttcttctaaccaaccaccggacacgacaggggctggaggcttgcctctcagtgctgagaggggggaaaggtcgccagccatgtactcacattcgtacatatggtcagtttagaactgacaattaacctaacatgaaagaaactggtgtatccagtatcccatggatgcaaactctgcacacacaggataatatgaaacttctaaatgttaaaaatgcatatgggaactttaatatttgtctttaaaggcagacagtggtgacactctggctcttctgtcagtgttttggttttcttttctaaattatgactgtggccttgtttgattttgttatgcagcggtgagcaccttcttccatgccagctgtgcccctggggcagcaagaggcagcaagctgtgtgaactgtgcaagggagactgctccaggtcccagagggagccttactatgactacaatggagcctttcagtcagtgtccactttttaatgcttctcattagtgaacataaacttctacacatttagccgaaccttttatctgtaataatatgacttttgtaacaggtgtttggccgagggagctggagatgtggcttttgtgaagcatctcactgtgccaggtcagtatcacaacaacctttatccattcgaacatataaagaagcactgcatcatagcattgtcaggttttattcatcaaaaacgagcctcaccgagattaaaaatttcattttgaaaagcgactgggacaagaagggacctgatgaacatgcatgacatacaaatacaactgttgcacactgcacaagtcaaggacaagtcaagcagaatttcaaagcaaatttcagagcaatcacattgaccaagagtgtcattttctcaaatgattaaatggtcttaaacaccctgaaacttctacaaatatgggaaagaataactgtgaaaaacactgatgtaaaacctgtgtcatgtcacacttccagaatcggataagccaatgtacgagctactgtgcaaggacaacaccagagcacctattgacaactataacacctgcttcctggccagagtgccaggtgacgctgttattactcgcaaggatccacagctggccgactttatctgggagacccttgaccgagttcagactgaccacgtaggaagtccactgaataacatctcatttcaacgtattctgattctttgtgtttcagcagcctcattgctttcctaatgaatcatctcacttgctgccttttcagagctttaacctcttctcatctgaagcctacgcgcccaccaagaacctgatgttcaaagattcaacagtgaagctcgtgagggtgcccccaaacacagactccttcctgtatctgggtgctaactacatgagcatcgtccattcccttaaaaaaggtaaagaacacaagcaagacaggcagaaacaattgttagtcaggtgtcggtctggcttatgtctaagtctttttcccgtctctagagcaggcatcagacgttgcatcccctgccattaggtggtgtgctgtgggccatgctgagactgctaagtgtgacacgtggagcatcaacagtgtgtctgatgacaccgcctccattgaatgccagagcgcccctacagttgaagattgcctgaagaagattatggtaaactcagtaggaaatatcgaaatctgttactttattattttttgactccactctgtgacgttcatctctgctttgaatcgccccgcagcgtaaagaggctgacgcagtcgccgtggatggaggtcaggtgtttacggctggaaagtgtggcctggttcctgtcatggtggagcagtatgaccaaggtagataacaagaagaagaaataacacacacacaatcctgtgtcgtatttcaccttttctatatgagtaatatttgttcattttgtcttctctcgcagagatgtgcggcaactctaatggtagtaaactttcatctttttcctcctctttttatgatctggacaaattttttaaaaagcctcttgaagtgcatcattttaattgtaacacagcttcgatgacctgttgtgctgatgacaagatttgtattttatatttgtatccttggactctaccactctactactacaactacaactataatttatatgaacaaccagttcaaggaatccttatttattcattagtaaccccttagttcatcctctgaaacaaacctctgcggaagctttgaacaacaggtggatggagcttgtcttcattatatattaaggatatagtctctttatggctttacaaacaaccaagatttttaaaaactacaaaaaaattccaaattgagcatttacataaatctggagacctagcatttagctcattggcattgcttgtacattgtctaaatttttagtggctccggttttgtaagtatccacgattttaatgatagattacctttaaaaagtggattaattttaagcagccgtaaacagagtagatttatatgaacgtctctctccagttgaatcatctgaaatgtctacttcccgtatccactctcagccccagcctcctcatactatgctgttgctgtggtaaagaagggttcaggggtgacctgggagaatctgaagggcaagaagtcttgccacacaggcatcggcagaactgctggctggaacatccccatgggtctaatctacaacagagaacatgactgtgacttcagtgagtgactgcgagcttcttccttcttttaccaacagaaagaaatttaaagccacccacagaaatcatacaactcccctttttctaaaaaaaaagtttgtttagatgactcatgtgattaatctatatttaaacaaaagccacaatcttccattaaccttgacaaaagagaggtttgctgattgttgagctctttctgcatctcttcctctcctcagctaagttcttcagtagcggctgtgcccccggagcagagcccacctctccgttctgttctctgtgtgtcggcagcggtaaagctgtgggagacgaggccaaatgcaaagccagcgctgatgagaagtactacggttatgctggagcctttaggtacagcaagamacagaatggcatcgcatacaagtttgtgtataaggagaaagttagcaaagagacaggaagcactggaaaacaacagaaccagctcttctsaatsttggcagctactgttatgttttttttcttatatatttcatttgtaaacagccaagaatgtgtcaagagctgaagcatgttttcctcctaaaaccaggactttttaatctttttttaaattgttgtgaactttagcaatgatgaggtgtaatttttaattggttagccatttacccccaacttcactctttgtctccaaagtcatgatgtatggcatatcattacaggtgtctggttgagggtggtggtgatgttgccttcattaaacacacaactgtgacagaaaacagcgatggtaagaggactgcactgatctgcactacctgctccaatgacattacaacagcttctaaacattattccacctctctttaccgaaggtaacggtccagactgggctcgtaatctgagatcttctgattaccagctcatctgccctgggaagggaccagtggagatcagtgattatgccacttgtaacttggctcaggtgcctgcacacgctgtggtgactcgcccagaaagccacagtaaggtggtccgcatccttcaggaacagcaggtttgtgataaagtctaagatagcyacttgaaatgaaataagagsttaaaataaaattgttgttgtttcaaagcactgaattcctagtgcaaaaatctkacatcrmggttcaacttttctagctgacacagatgctgagtctgaaatagagaagcatcctgtcattacagaagctgcatacagtttgtttggcattctgctttgagcacaacaatgaatttttaacagattttccaaatagttgccaattaactctgacaactttaacattgtccctttgtaggggctacatctaagactctacaggcctcagttaacatgttaaatgttaaagcttatgacagcagaattagaaaaagactgagcaaatgtagcttgtttggaaagaaaaagccgcttctctctacagagaacatggcagcacagcttttgtttgcagagttgcatctgaacaaaccaagagacttctggaacaatgttcgtctggccttaatgtacaggaccacgtttgttgaaaatcaaacatagcatattcgcacaaacacctcatagaaactggtggtggaggggtgttgatttgggcttcttttacagcaacgtggcacaaacatttcaagttactgctcccaaagttgattctacttgctactgaatcatggagtgtacttagtttgtcacaactgcatagagtcctgtgaaaactttctttttgatatgactgtacttgcacttgatctcaagtggggggaccttggcaattcaaaaaccaatagtgacttctgtttttctacattctgccggtttcaatgagtaaagtaaccagtgtaatagaaatctaaaaattaaataactgcttcaaacttaaattttgttcataggctagatttggaaacagcgggactgatccttcattcagaatgttccagtcagagaatggaaagaaccttctcttccaagactccactaagtgtctccaggaggtaccagagggaagaacctatgagcagtttttgggaatttcttacatgaccgccatgaaggcactgagacattgcagcgaaactactccaggtttgtactactgcagaaattacttagcacttaaatgttacttttgtaccatcttgtaccagataaaataacattcatgctcgatattctccaattgtttttgtttgcatctcttcccaacagatctggaaaagtcttgcacattccatgcctgccagcaaggctaatggagatgtagacaacacagtgattcagccatcttacccacttacttcctgcacttacccaaacatagtgtttctcctcttctgatcttactatagactataga

SEQ ID NO: 15 - protein, 694 aa, Tilapia nilotica
MKPLLLVALLGLLGKEFAAPGSQKVKWCVKSDQELRKCSDLAAAIPCVICVKKENTLECIVAIKAGEADAITVDGGDIYTAGLNNYDLHPIIAEDYGTASDTCYYAVAVAKKNTGFGFRDLRGKKSCHTGLGKSAGWNIPIGSLVSMNVIEWGGIEDKPLEEAVSTFFHASCAPGAARGSKLCELCKGDCSRSQREPYYDYNGAFQCLAEGAGDVAFVKHLTVPESDKPMYELLCKDNTRAPIDNYNTCFLARVPGDAVITRKDPQLADFIWETLDRVQTDHSFNLFSSEAYAPTKNLMFKDSTVKLVRVPPNTDSFLYLGANYMSIVHSLKKEQASDVASPAIRWCAVGHAETAKCDTWSINSVSDDTASIECQSAPTVEDCLKKIMRKEADAVAVDGGQVFTAGKCGLVPVMVEQYDQEMCGNSNAPASSYYAVAVVKKGSGVTWENLKGKKSCHTGIGRTAGWNIPMGLIYNREHDCDFTKFFSSGCAPGAEPTSPFCSLCVGSGKAVGDEAKCKASADEKYYGYAGAFRCLVEGGGDVAFIKHTTVTENSDGNGPDWARNLRSSDYQLICPGKGPVEISDYATCNLAQVPAHAVVTRPESHSKVVRILQEQQTRFGNSGTDPSFRMFQSENGKNLLFQDSTKCLQEVPEGRTYEQFLGISYMTAMKALRHCSETTPDLEKSCTFHACQQG

(19) United States
(12) Patent Application Publication
(10) Pub. No.: US 2009/0165156 A1
(43) Pub. Date: Jun. 25, 2009

(54) METHOD FOR INCREASING THE SALTWATER TOLERANCE OF A FISH

(75) Inventors: Aina Haugen Rengmark, Oslo (NO); Frode Lingaas, Fagernes (NO)

Correspondence Address: WOLF GREENFIELD & SACKS, P.C., 600 ATLANTIC AVENUE, BOSTON, MA, 02210-2206, US

(73) Assignee: Norwegian School of Veterinary School, Oslo (NO)

(21) Appl. No.: 12/258,105

(22) Filed: Oct. 24, 2008

Related U.S. Application Data
(63) Continuation-in-part of application No. PCT/GB2007/002291, filed on Jun. 20, 2007.
(60) Provisional application No. 61/000,324, filed on Oct. 24, 2007.

(30) Foreign Application Priority Data
Jun. 20, 2006 (NO) NO2006/2887

(51) Int. Cl. C12N 15/00; C12Q 1/68; C12Q 1/00; C07K 16/00; C07K 14/00; C07H 21/04; C12N 15/63

(52) U.S. Cl. 800/22; 435/6; 435/4; 530/387.9; 530/350; 536/23.1; 435/320.1

(57) ABSTRACT

The present invention relates to a method for obtaining an indication of the saltwater tolerance of a fish, and methods for increasing the saltwater tolerance of a fish. In particular, the invention relates to methods involving biomarkers in the transferrin gene which are correlated with high or low saltwater tolerance.

### RELATED APPLICATIONS

This applications claims priority under 35 U.S.C. §119(e) from U.S. provisional application Ser. No. 61/000,324, filed Oct. 24, 2007, and is a continuation-in-part of PCT application PCT/GB2007/002291, filed Jun. 20, 2007, which claims priority from Norwegian application No. 20062887, filed Jun. 20, 2006, the entire contents of each of which are incorporated herein by reference.

### FIELD OF THE INVENTION

The present invention relates to a method for obtaining an indication of the saltwater tolerance of a fish, and methods for increasing the saltwater tolerance of a fish.

### BACKGROUND OF THE INVENTION

Tilapias belong to a genus of fish within the cichlid family and are becoming the world's leading aquaculture species, with the Nile tilapia (Oreochromis niloticus) at the forefront (Bentsen et al., 1998; Kocher, 2002; Roderick, 1999; Trewavas, 1983).

Tilapias are easily raised and harvested, may be fed a diet of abundant algae and zooplankton. The commercially most important species and strains are predominantly found in fresh water. Since the availability of freshwater is severely limited in many countries, the exploitation of brackish water areas and other facilities, typically abandoned coastal shrimp farms, may present an opportunity to expand the tilapia aquaculture industry with only modest investments (Yi et al., 2002). However, such development requires a tilapia that tolerates saltwater without reduction in growth performance and health. Increased knowledge of genes involved in saltwater tolerance may facilitate selection for this trait.

The genus Oreochromis tolerates brackish water, but with a great variation among the species. Oreochromis mossambicus and O. aureus (blue tilapia) show a higher degree of salt tolerance than the Nile tilapia (Avella et al., 1993; Cataldi et al., 1988; Doudet, 1992), indicating that salt tolerance may be controlled by genetic factors.

WO03/018845 describes a method for identifying fast-growing fish in different salinities using a selection method based on their prolactin-1 genotype.

### SUMMARY OF THE INVENTION

The present invention relates to a method for obtaining an indication of the saltwater tolerance of a fish, and methods for increasing the saltwater tolerance of a fish. It has now been found that a number of biological markers in the transferrin gene are correlated with saltwater tolerance.

In some embodiments, methods of determining the saltwater tolerance of a fish are provided, the method comprising determining the status of a biomarker in a fish, wherein the biomarker is one which is correlated with high or low saltwater tolerance of the fish, and wherein the presence or absence of said biomarker is indicative of the saltwater tolerance of the fish.

In certain embodiments, the biomarker is transferrin DNA, RNA or polypeptide, the glycosylation pattern of the transferrin polypeptide, or the iron-binding ability of the transferrin polypeptide.

In certain embodiments, the biomarker is a variation of the nucleotide sequence of the DNA in or near the transferrin gene, a variation of the nucleotide sequence of the transferrin RNA, a variation of the amino acid sequence of the transferrin polypeptide.

In certain embodiments, the biomarker is one or more single nucleotide polymorphisms (SNP), a haplotype, a microsatellite, an alternatively-spliced RNA product, or a polypeptide epitope.

In certain embodiments, the biomarker is one or more single nucleotide polymorphism (SNP), and wherein the one or more SNP is at a position in the transferrin gene selected from the group consisting of: ex7 (bp2968), ex8-1 (bp3190), ex8-2 (bp3224), ex8-3 (bp3229), intr8 (bp3330), ex14 (bp5368), intr14-1 (bp5437), intr14-2 (bp5462-3), intr14-3 (bp5467), ex15-1 (bp5521), ex15-2 (bp5598), intr15-1 (bp5689), intr15-2 (bp6530), ex16-1 (bp6549), ex16-2 (bp6562-4), ex16-3 (bp6659), ex16-4 (bp6685-8), ex16-5 (bp6697-9), ex16-6 (bp6729), ex16-7 (bp6733), ex16-8 (bp6736), intr16-1 (bp6769), intr16-2 (bp6777), intr16-3 (bp6780), intr16-4 (bp6785), intr16-5 (bp6848).

In some embodiments, a DNA, RNA or polypeptide sample is obtained from the fish, wherein the biomarker is correlated with high saltwater tolerance of the fish, and wherein the presence of said biomarker in the sample is indicative of high saltwater tolerance of the fish.

In certain embodiments, the DNA in the sample carrying the variation of the nucleotide sequence in or near the transferrin gene is amplified.

In some embodiments, methods of determining the saltwater tolerance of a fish are provided, the method comprising (a) obtaining a sample from the fish comprising transferrin polypeptide, (b) contacting the sample with an antibody that specifically binds to a transferrin polypeptide comprising one or more of the amino acid changes of haplotype 2, selected from the group consisting of Ala256 (A)→Gly256 (G), Ala295 (A)→Thr295 (T), Arg306 (R)→Lys306 (K), Leu308 (L)→Val308 (V), Val545 (V)→Ile545 (I), Ala593 (A)→Val593 (V), Ala617 (A)→Thr617 (T), Ala622 (A)→Ser622 (S), Glu654 (E)→Gly654 (G), Glu663 (E) Ala664 (A)→Ile663 (I) Ser664 (S), Asp667 (D)→Thr667 (T), Asp677 (D)→Glu677 (E), Ala679 (A)→Thr679 (T), and Ser680 (S)→Pro680 (P), and (c) detecting the binding of the antibody, wherein binding of the antibody indicates the saltwater tolerance of the fish.

In some embodiments, methods for increasing the saltwater tolerance of a fish are provided, the method comprising integrating a transferrin gene stably into the genome of the fish in a position such that the transferrin gene is expressed in some or all tissues of the fish, wherein expression of transferrin increases the saltwater tolerance of the fish.

In certain embodiments, the methods further comprise selecting a male fish from the population and obtaining sperm from the male fish.

In certain embodiments, the methods further comprise selecting a female fish from the population and obtaining eggs from the female fish.

In certain embodiments, the methods further comprise breeding the saltwater tolerant fish of any of the preceding methods with a fish of the opposite sex in order to produce progeny fish.

In certain embodiments, the methods further comprise obtaining gametes from the saltwater tolerant fish of any of the preceding methods, combining the gametes from the saltwater tolerant fish with gametes from a second fish of the opposite sex in order to produce progeny fish and selecting progeny which have high saltwater tolerance.

In certain embodiments, the fish is a teleost or bony fish.

In certain embodiments, the fish is a Cichlidae, Salmonidae, Cyprinidae or Gadidae.

In some embodiments, isolated antibodies are provided, wherein the antibody specifically binds to a transferrin polypeptide comprising one or more of the amino acid changes of haplotype 2, selected from the group consisting of Ala256 (A)→Gly256 (G), Ala295 (A)→Thr295 (T), Arg306 (R)→Lys306 (K), Leu308 (L)→Val308 (V), Val545 (V)→Ile545 (I), Ala593 (A)→Val593 (V), Ala617 (A)→Thr617 (T), Ala622 (A)→Ser622 (S), Glu654 (E)→Gly654 (G), Glu663 (E) Ala664 (A)→Ile663 (I) Ser664 (S), Asp667 (D)→Thr667 (T), Asp677 (D)→Glu677 (E), Ala679 (A)→Thr679 (T), and Ser680 (S)→Pro680 (P).

In some embodiments, isolated polypeptides comprising the amino acid sequence of SEQ ID NO: 15 are provided.

In certain embodiments, isolated nucleic acid molecules are provided, encoding the polypeptide of SEQ ID NO: 15 or comprising the nucleic acid sequence of SEQ ID NO: 14.

In certain embodiments, vectors or plasmids are provided comprising the nucleic acid molecules encoding the polypeptides of SEQ ID NO: 15 or comprising the nucleic acid molecules comprising the nucleic acid sequence of SEQ ID NO: 14.

In some embodiments, methods of obtaining an indication of the saltwater tolerance of a fish are provided, the methods comprising establishing the presence or absence of a biomarker in or near the transferrin gene of the fish, in the transferrin RNA or in the transferrin polypeptide of the fish, wherein the biomarker is one which is correlated with high or low saltwater tolerance of the fish.

In some embodiments, the biomarker is DNA sequence information, one or more single nucleotide polymorphisms (SNPs), haplotypes, microsatellites, RNA-variants, including alternatively-spliced products, amino acid sequence information, polypeptide epitopes, glycosylation patterns or the iron-binding ability of the transferrin polypeptide.

In some embodiments, methods of obtaining an indication of the saltwater tolerance of a fish are provided, the methods comprising establishing the presence or absence in a DNA sample obtained from the fish of a biomarker in or near the transferrin gene wherein the biomarker is one which is correlated with high saltwater tolerance of the fish and wherein the presence of said biomarker in the DNA sample is indicative of high saltwater tolerance of the fish.

In some embodiments, methods of obtaining an indication of the saltwater tolerance of a fish are provided, the method comprising establishing the presence or absence in a DNA sample obtained from the fish of at least one single nucleotide polymorphism (SNP) in the transferrin gene, wherein at least one of the SNPs is at a position in the transferrin gene which corresponds to a position of a SNP shown in Table 2 and/or Table 3.

In some embodiments, methods of obtaining an indication of the saltwater tolerance of a fish are provided, the method comprising establishing the presence or absence in a biological sample obtained from a transferrin-expressing tissue from the fish of a biomarker in the transferrin RNA wherein the biomarker is one which is correlated with high salt water tolerance in the fish, wherein the presence of said biomarker in the biological sample is indicative of high saltwater tolerance of the fish.

In some embodiments, methods of obtaining an indication of the saltwater tolerance of a fish are provided, the method comprising establishing the presence or absence in a biological sample obtained from a transferrin-expressing tissue from the fish of at least one single nucleotide polymorphism (SNP) in the transferrin RNA, wherein at least one of the SNPs is at a position in the transferrin RNA which corresponds to a position of a SNP shown in Table 2 and/or Table 3.

In certain embodiments, all preceding methods are provided, further comprising the step of amplifying the region of the fish DNA which includes the biomarker in question.

In some embodiments, kits comprising at least one nucleic acid primer are provided, wherein the nucleic acid primer is capable of hybridising under high stringency conditions to the coding or non-coding strand of a region of DNA in or near the transferrin gene and which can be used in an exponential amplification reaction to amplify a region of DNA which comprises at least one of the microsatellites given in Table 2 or 3, or at least one of the SNPs in Table 2 or 3, or corresponding SNPs or microsatellites in the fish in question.

In some embodiments, methods of obtaining an indication of the saltwater tolerance of a fish are provided, the method comprising determining the presence in the fish of a transferrin polypeptide isoform which is associated with high salt water tolerance.

In some embodiments, methods of obtaining an indication of the saltwater tolerance of a fish are provided, the method comprising detecting whether a first antibody binds to a transferrin-containing sample obtained from the fish, wherein said first antibody is an antibody which binds specifically to a transferrin polypeptide which has one or more of the amino acid changes of Haplotype 2 at the positions shown in Table 2 or Table 3 or at corresponding positions in the transferrin polypeptide of the said fish, wherein the antibody does not bind to a transferrin polypeptide which does not have any of the amino acid changes of Haplotype 2 at the positions shown in Table 2 or Table 3 or at corresponding positions in the transferrin polypeptide of the said fish, and/or detecting whether a second antibody binds to a transferrin-containing sample obtained from the fish, wherein said second antibody is an antibody which binds specifically to a transferrin polypeptide which does not have any of the amino acid changes of Haplotype 2 at the positions shown in Table 2 or Table 3 or at corresponding positions in the transferrin polypeptide of the said fish, wherein the second antibody does not bind to a transferrin polypeptide which has one or more of the amino acid changes of Haplotype 2 at the positions shown in Table 2 or Table 3 or at corresponding positions in the transferrin polypeptide of the said fish.

In some embodiments, antibodies are provided, wherein the antibodies bind specifically to a transferrin polypeptide which has one or more of the amino acid changes of Haplotype 2 at the positions shown in Table 2 or Table 3 or at corresponding positions in the transferrin polypeptide of the said fish, wherein the antibodies do not bind to a transferrin polypeptide which does not have any of the amino acid changes of Haplotype 2 at the positions shown in Table 2 or Table 3 or at corresponding positions in the transferrin polypeptide of the said fish; or antibodies that bind specifically to a transferrin polypeptide which does not have any of the amino acid changes of Haplotype 2 at the positions shown in Table 2 or Table 3 or at corresponding positions in the transferrin polypeptide of the said fish, wherein the antibodies do not bind to a transferrin polypeptide which has one or more of the amino acid changes of Haplotype 2 at the positions shown in Table 2 or Table 3 or at corresponding positions in the transferrin polypeptide of the said fish.

In some embodiments, kits are provided comprising an antibody which binds specifically to a transferrin polypeptide which has one or more of the amino acid changes of Haplotype 2 at the positions shown in Table 2 or Table 3, wherein the antibody does not bind to a transferrin polypeptide which does not have any of the amino acid changes of Haplotype 2 at the positions shown in Table 2 or Table 3, and/or an antibody which binds specifically to a transferrin polypeptide which does not have any of the amino acid changes of Haplotype 2 at the positions shown in Table 2 or Table 3, wherein the antibody does not bind to a transferrin polypeptide which has one or more of the amino acid changes of Haplotype 2 at the positions shown in Table 2 or Table 3, and optionally, appropriate instructions for use.

In some embodiments, methods of finding a biomarker which is indicative of saltwater tolerance in a fish are provided, the methods comprising determining the presence in or near the transferrin gene of a biomarker which is present in fish which are saltwater tolerant but is not present in fish which are not saltwater tolerant.

In some embodiments, methods of finding a biomarker other than transferrin which is indicative of saltwater tolerance in a fish are provided, the methods comprising providing a population of fish which have a first biomarker in or near the transferrin gene of the fish, wherein the first biomarker is one which is correlated with saltwater tolerance of the fish, establishing the presence in a DNA, RNA or polypeptide sample of the fish of a second biomarker which is correlated with the presence of the first biomarker, wherein the second biomarker is not in or near the transferrin gene, in the transferrin RNA or in the transferrin polypeptide, wherein the second biomarker is one which is indicative of salt tolerance of the fish.

In some embodiments, methods of producing a fish are provided, the methods comprising selecting a first fish whose saltwater tolerance has been determined by any suitable method described herein, and breeding said first fish with a fish of the opposite sex in order to produce progeny fish.

In some embodiments, methods of producing a fish are provided, the methods comprising determining the saltwater tolerance of a first fish by any suitable method described herein, and breeding said first fish with a fish of the opposite sex in order to produce progeny fish.

In some embodiments, methods of producing a fish are provided, the methods comprising selecting a first fish whose saltwater tolerance has been determined by any suitable method described herein, obtaining gametes from the first fish, and combining the gametes from the first fish with gametes from a second fish of the opposite sex in order to produce progeny fish.

In some embodiments, methods of producing a fish are provided, the methods comprising determining the saltwater tolerance of a first fish by any suitable method described herein, obtaining gametes from the first fish, combining the gametes from the first fish with gametes from a second fish of the opposite sex in order to produce progeny fish.

In some embodiments, methods of producing a fish are provided, the methods comprising determining the saltwater tolerance of a population of fish by any suitable method described herein, selecting two fish from the population, wherein the two fish are of opposite sex and both have high saltwater tolerance, breeding the two fish of opposite sex in order to produce progeny fish, and selecting progeny which have high saltwater tolerance.

In some embodiments, methods of producing a transgenic fish are provided, the methods comprising integrating a transferrin gene stably into the genome of the fish in a position such that the transferrin gene is expressed in some or all tissues of the fish.

In some embodiments, transgenic fish are provided, wherein the fish comprises a heterologous transferrin gene stably-integrated into its genome in a position such that the transferrin gene is expressed in some or all tissues of the fish.

In some embodiments, methods of increasing the saltwater tolerance of a fish are provided, comprising introducing into the fish an agent which increases the transferrin levels in the fish.

In some embodiments, methods of producing fish sperm are provided, comprising determining the saltwater tolerance of a population of fish by any suitable method described herein, selecting a male fish from the population; and obtaining sperm from the male fish.

In some embodiments, methods of producing fish eggs are provided, comprising determining the saltwater tolerance of a population of fish by any suitable method described herein, selecting a female fish from the population; and obtaining eggs from the female fish.

In some embodiments, any of the preceding methods are provided, wherein the fish is a teleost or bony fish.

In some embodiments, any of the preceding methods are provided, wherein the fish is a Cichlidae, Salmonidae, Cyprinidae or Gadidae.

In some embodiments, nucleic acid molecules whose nucleotide sequence comprises the sequence of SEQ ID NO: 14 or which encodes the amino acid sequence of SEQ ID NO: 15 are provided.

In certain embodiments, vectors or plasmids are provided comprising the nucleic acid molecules of SEQ ID NO: 14, or nucleic acid molecules that encode the amino acid sequence of SEQ ID NO: 15.

In certain embodiments, polypeptides are provided, wherein the amino acid sequence of the polypeptide consists of, or comprises the amino acid sequence of SEQ ID NO: 15.

### BRIEF DESCRIPTION OF FIGURES

FIG. 1: Protein blast result (by Mega) with Nile tilapia, black and red sea bream, Japanese medaka and brown trout. The different symbols are as follows: identical amino acid (.), indels in sequence alignments (-) and stop codon (*). Marked in bold: conserved iron and anion binding residues (Lambert et al., 2005). The numbering are different than the notation used in Lambert et al. (2005) since the human protein was used as a model and the numbering there starts after the signal peptide cleavage at amino acid position 19 (similar for all the species included). The insertion of three amino acids in Nile tilapia only is underlined, so are the SNPs that cause amino acid change.

### DESCRIPTION OF SEQUENCES

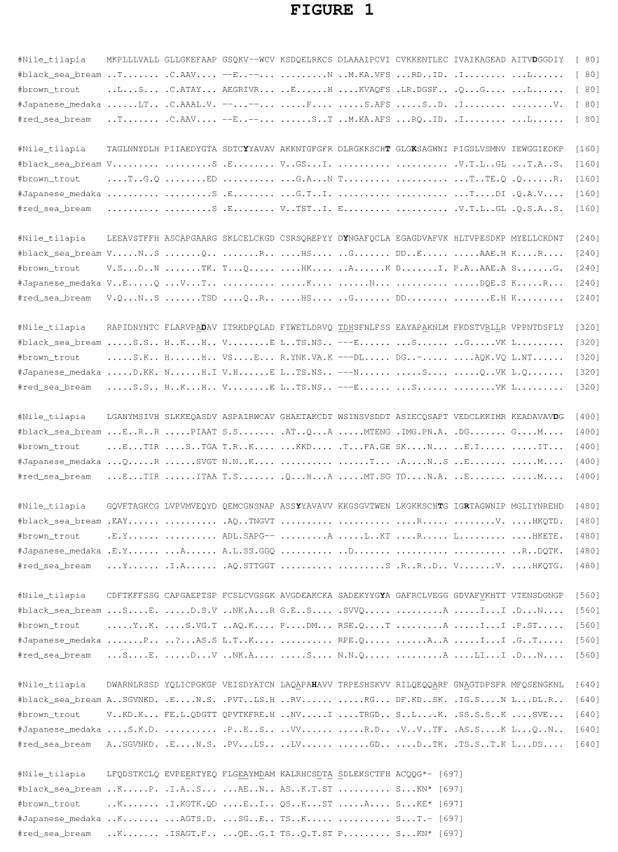

SEQ ID NO: 1 is the low saltwater tolerance form of the nucleotide sequence of the nile tilapia transferrin gene.
SEQ ID NO: 2 is the low saltwater tolerance form of the amino acid sequence of the nile tilapia transferrin polypeptide.
SEQ ID NO: 3 is the nucleotide sequence of the microsatellite region TF-A.
SEQ ID NO: 4 is the nucleotide sequence of the microsatellite region TF-B.
SEQ ID NO: 5 is the nucleotide sequence of the first PCR primer used to amplify microsatellite region TF-A.
SEQ ID NO: 6 is the nucleotide sequence of the second PCR primer used to amplify microsatellite region TF-A.
SEQ ID NO: 7 is the nucleotide sequence of the first PCR primer used to amplify microsatellite region TF-B.
SEQ ID NO: 8 is the nucleotide sequence of the second PCR primer used to amplify microsatellite region TF-B.
SEQ ID NO: 9 is the amino acid sequence of the Tilapia nilotica polypeptide, as given in FIG. 1.
SEQ ID NO: 10 is the amino acid sequence of the Acanthopagrus schlegeli polypeptide, as given in FIG. 1.

SEQ ID NO: 11 is the amino acid sequence of the Salmo trutta polypeptide, as given in FIG. 1.

SEQ ID NO: 12 is the amino acid sequence of the Oryzias latipes polypeptide, as given in FIG. 1.

SEQ ID NO: 13 is the amino acid sequence of the Pagrus major polypeptide, as given in FIG. 1.

SEQ ID NO: 14 is the high saltwater tolerance form of the nucleotide sequence of the nile tilapia transferrin gene, i.e., SEQ ID NO: 1 with all of the SNPs of Haplotype 2 as shown in Tables 2 and 3.
SEQ ID NO: 15 is the high saltwater tolerance form of the amino acid sequence of the nile tilapia transferrin polypeptide, i.e., SEQ ID NO: 2 with all of the SNPs of Haplotype 2 as shown in Tables 2 and 3.

### DETAILED DESCRIPTION OF THE INVENTION

It has now been found that a number of biological markers in the transferrin gene are correlated with saltwater tolerance.

Transferrin is an iron binding glycoprotein. It is involved in several biological functions in a wide range of organisms, primarily as an iron transporter. Transferrin also has an important role in the immune system, since the binding of iron limits the availability of iron for replicating pathogens (Cnaani et al., 2002; Stafford and Belosevic, 2003). Transferrin (TF) is expressed primarily in liver and transported around the body in plasma supplying most body-tissues with iron, but is also expressed in several other organs (Briggs et al., 1999). Experiments show that the gene is expressed in the brain in Atlantic cod (Gadus morhua) in contrast to salmon and other vertebrates where brain expression was not detected (Denovan-Wright et al., 1996).

Transferrin belongs to a gene family including ovotransferrin (OTF) and lactotransferrin (LTF). OTF is encoded by the avian transferrin gene and is expressed in eggs and LTF is secreted into milk by the mammary gland preventing proliferation of invading microbes. These variants of transferrin are believed to be produced locally (in mammals and avian) not readily reached by transferrin in plasma (Briggs et al., 1999; Lambert et al., 2005).

The invention provides simple and easy methods for determining the saltwater tolerance of organisms via the use of biomarkers in the transferrin gene.

In one embodiment, the invention provides a method of obtaining an indication of the saltwater tolerance of a fish, the method comprising establishing the presence or absence of a biomarker in or near the transferrin gene of the fish, in the transferrin RNA or in the transferrin polypeptide of the fish wherein the biomarker is one which is correlated with high or low saltwater tolerance of the fish.

In some embodiments of the invention, the term “obtaining an indication of” as used herein means “contributing to the prediction of.” Accordingly, obtaining an indication of the saltwater tolerance of a fish may involve evaluating, predicting and/or determining whether it is saltwater tolerant and/or its level of saltwater tolerance. In some embodiments, the term “establishing the presence or absence of a biomarker” means establishing whether the biomarker is present or absent. Accordingly, establishing the presence or absence of a biomarker may involve using one or more detection and/or analytical techniques to determine whether one or more biomarkers is present or absent (e.g., as described herein). Accordingly, methods of the invention may be used to identify, select or isolate one or more fish (e.g., fish populations) that are saltwater tolerant or that have a desired level of saltwater tolerance.

In the context of the present invention, the term “fish” covers teleost or bony fish, and cartilagenous fish. Teleost or bony fish are preferred. More preferably, the teleost fish is a Cichlidae (e.g., tilipia), Salmonidae (e.g., altantic, coho, and rainbow salmon), Cyprinidae (e.g., carp) or Gadidae (e.g., cod). Particularly preferably, the fish is a Tilapia, (Oreochromis), such as O. niloticus (nile tilapia), O. mossambicus or O. aureus (blue tilapia).

In some embodiments, the fish is a freshwater fish or primarily a freshwater fish. In other embodiments, the fish is a saltwater fish or primarily a saltwater fish. In yet other embodiments, the fish is one which has an ability to change between living in freshwater and saltwater in its natural life cycle.

The person skilled in the art will readily be able to establish the location and/or nucleotide sequence of the transferrin gene in the fish being tested. Sequences of fish transferrin genes are known in the art. The sequence of nile tilapia transferrin gene is shown herein as SEQ ID NO: 1. Comparisons between known transferrin genes and putative transferrin genes from the fish in question can be made by standard means. This may be done either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/), the Megalign program of the LASERGENE bioinformatics computing suite (DNASTAR Inc., Madison, Wis.) or using the Clustal method of alignment (Higgins and Sharp (1989) CABIOS. 5: 151-153). If using the Clustal method, default parameters for pairwise alignments may be KTUPLE 1, GAP PENALTY=3, WINDOW=5 and DIAGONALS SAVED=5. Sequences with at least 80%, preferably more than 90% or more than 95% sequence identity with SEQ ID NO: 1 (under the above-mentioned parameters) and which have transferrin activity (e.g., ability to bind iron) can be considered to be transferrin genes.

The person skilled in the art will be aware of the existence of other members of the transferrin family of genes, e.g., lactotransferrin and ovotransferrin. In some embodiments of the invention, the invention relates to corresponding methods, products, uses and processes, etc., that involve the use of lactotransferrin or ovotransferrin in place of transferrin, mutatis mutandis.

The biomarker may be present “in or near the fish transferrin gene.” In the present context, the term “transferrin gene” is intended to include all regions of DNA from and including the transferrin promoter, untranslated 5′ sequence, the transferrin introns and exons, and the 3′ terminator sequence.

The term “near the transferrin gene” is intended to cover regions of DNA which co-segregate with the transferrin gene and hence which can provide biomarkers which co-segregate with the transferrin gene. In this context, the term “near the transferrin gene” may, for example, be less than 30 centimorgan (cM), preferably less than 20 cM and most preferably less than 10, 5, 2 or 1 cM from the coding sequence of the transferrin gene. The skilled person will understand that the closer the biomarker is to the coding sequence of the transferrin gene, the greater the probability will be that the biomarker and the transferrin gene will co-segregate in any one fish.

In the context of the above method, the biomarker may be any marker which can be correlated with the degree of saltwater tolerance of the fish, i.e. which can be used to distinguish between fish having a high or low saltwater tolerance. Examples of suitable biomarkers include DNA sequence information, one or more single nucleotide polymorphisms (SNPs), haplotypes, microsatellites, RNA-variants, including alternatively-spliced products, amino acid sequence information, polypeptide epitopes, glycosylation patterns and the iron-binding ability of the transferrin polypeptide. The correlation is preferably a significant one, e.g. p<0.05, 2-tailed test.

In the context of the present invention, the term “low saltwater tolerance” or “not saltwater tolerant” is intended to mean that the organism is not capable of surviving 3 days in water comprising 30 ppt salt or higher salt concentration; and the term “high saltwater tolerance” or “saltwater tolerant” is intended to mean that the organism is capable of surviving 3 days in water comprising 30 ppt salt. As used herein, the term “ppt” means parts per thousand; and the term “salt” refers to sodium chloride, i.e., sea salt.

The Examples described herein demonstrate a significant correlation between the presence of a number of biomarkers in or near the transferrin gene of a fish and high or low saltwater tolerance in the fish. It can be seen from these Examples therefore that, in order to obtain an indication of the saltwater tolerance of the fish, it is not necessary to establish whether or not the fish is homozygous or heterozygous for the biomarker in question. However, establishing whether a fish is homozygous or heterozygous for the marker in question will provide a higher degree of confidence in the indication of saltwater tolerance.

In a further embodiment, the invention provides a method of obtaining an indication of the saltwater tolerance of a fish, the method comprising establishing the presence or absence in a DNA sample obtained from the fish of a biomarker in or near the transferrin gene wherein the biomarker is one which is correlated with high saltwater tolerance of the fish and wherein the presence of said biomarker in the DNA sample is indicative of high saltwater tolerance of the fish.

Through the typing of these biomarkers associated with saltwater tolerance, the individuals can be defined as homozygous for a haplotype variant (e.g., Hap1/Hap1), heterozygous (e.g., Hap1/Hap2), or heterozygous for a second haplotype (e.g., Hap2/Hap2). The typing of the markers disclosed herein may also identify new haplotypes with different associations to saltwater tolerance. In some embodiments, it can be important to know if a breeder fish is homozygous or heterozygous for the beneficial haplotype. Homozygous individuals will give one beneficial haplotype to each of its offspring, while in the case of heterozygous breeder fish, they will just provide a beneficial haplotype to half of the offspring. This will strongly influence salt-tolerance and survival in the offspring groups.

The invention also provides a method of obtaining an indication of the saltwater tolerance of a fish, the method comprising establishing the presence or absence in a DNA sample obtained from the fish of a biomarker in or near the transferrin gene wherein the presence in the DNA sample of a first biomarker which is correlated with low saltwater tolerance of the fish and the absence of a second biomarker at the same position as the first (but on a different chromosome, i.e., an allele) which is correlated with high saltwater tolerance of the fish, i.e., the fish is homozygous for the first biomarker, is indicative of a low level of saltwater tolerance of a fish or indicative of a lower level of saltwater tolerance than a fish which is heterozygous for the first marker.

The term “sample” as used herein describes a biological sample containing genomic DNA, RNA and/or protein. Fin clips may be used, for example, to obtain DNA, RNA and/or protein. The DNA, RNA and/or protein may be extracted and purified by standard protocols.

In the context of the above method, the biomarker is a marker in the DNA which can be correlated with high or low saltwater tolerance. Examples of such biomarkers include DNA sequence information, SNPs, haplotypes and microsatellites, inter alia.

Methods of DNA sequencing are well established in the art. Examples of standard DNA sequencing methods include those based on techniques developed by Maxam and Gilbert (1977) or Sanger (1977). A variety of automated sequencing procedures may also be utilized, as may sequencing by mass spectrometry.

Preferred SNPs are given in Table 2 and Table 3. The SNPs shown in Table 2 and Table 3 are those that can be used to distinguish the saltwater tolerance phenotype of nile tilapia. The skilled person will readily understand, however, that SNPs at corresponding positions will also exist in the transferrin genes of other fish, and that those SNPS can also be used to distinguish salt water tolerance phenotypes of those fish.

In a preferred embodiment, the invention provides a method of obtaining an indication of the saltwater tolerance of a fish, the method comprising establishing the presence or absence in a DNA sample obtained from the fish of at least one single nucleotide polymorphism (SNP) in the transferrin gene, wherein at least one of the SNPs is at a position in the transferrin gene which corresponds to a position of a SNP shown in Table 2 and/or Table 3.

Preferably, the SNP(s) is one which is indicative of a high level of saltwater tolerance, i.e., one or more of the SNPs selected from those listed in Haplotype 2 in Tables 2 or 3 or the SNPs at corresponding positions in the fish in question. The presence of one or more of the aforementioned SNPs is indicative of high saltwater tolerance of the fish.

Preferably, the presence or absence of at least 2, 3, 4, 5, 10, 20 or all of the SNPs shown Table 2 or Table 3 is determined in the transferrin gene of the fish or the SNPs at the corresponding positions in the transferrin gene in the fish in question.

Microsatellite markers, i.e., regions of repeating DNA having a base core repeat unit, which are present in or near the transferrin gene have been found to co-segregate with the transferrin gene as one haplotype. Consequently, such microsatellite markers can be used as biomarkers to predict the saltwater tolerance of the fish.

Examples of suitable microsatelitte markers include (gt)ₙ, where n=8-20, preferably, 9-15, and most preferably 10-14. In certain preferred embodiments, n=10 or n=14.

In a further embodiment, the invention provides a method of obtaining an indication of the saltwater tolerance of a fish, the method comprising establishing the presence or absence in a biological sample obtained from a transferrin-expressing tissue from the fish of a biomarker in the transferrin RNA wherein the biomarker is one which is correlated with high salt water tolerance in the fish, wherein the presence of said biomarker in the biological sample is indicative of high saltwater tolerance of the fish.

The absence of a second biomarker at the same position as the first (but on a different RNA transcript, i.e., an allele) which is correlated with low saltwater tolerance of the fish, i.e., the fish is homozygous for the first biomarker, is indicative of a higher level of saltwater tolerance than a fish which is heterozygous for the first marker.

The invention also provides a method of obtaining an indication of the saltwater tolerance of a fish, the method comprising establishing the presence or absence in a biological sample obtained from a transferrin-expressing tissue from the fish of a first biomarker in the transferrin RNA which is correlated with low saltwater tolerance of the fish wherein the presence of the first biomarker and the absence of a second biomarker at the same position as the first (but on a different RNA transcript, i.e., an allele) which is correlated with high saltwater tolerance of the fish, i.e., the fish is homozygous for the first biomarker, is indicative of a low level of saltwater tolerance.

In a preferred embodiment, the invention provides a method of obtaining an indication of the saltwater tolerance of a fish, the method comprising establishing the presence or absence in a biological sample obtained from a transferrin-expressing tissue from the fish of at least one single nucleotide polymorphism (SNP) in the transferrin RNA, wherein at least one of the SNPs is at a position in the transferrin RNA which corresponds to a position of a SNP shown in Table 2 and/or Table 3.

Preferably, the SNP(s) is one which is indicative of a high level of saltwater tolerance, i.e., one or more of the SNPs selected from those listed in Haplotype 2 in Tables 2 or 3 or the SNPs at corresponding positions in the transferrin RNA in the fish in question. The presence of one or more of the aforementioned SNPs is indicative of high saltwater tolerance of the fish.

Preferably, the presence or absence of at least 2, 3, 4, 5, 10, 20 or all of the SNPs shown Table 2 or Table 3 is determined in the transferrin RNA of the fish or the SNPs at the corresponding positions in the transferrin RNA in the fish in question.

The RNA sample may be mRNA. Any tissue of the fish which expresses transferrin may be used. Transferrin is primarily expressed in the liver, but it is also expressed in several other organs, depending on the fish species. In tilapia, transferrin is expressed in brain, gills and liver, inter alia. The RNA may be extracted and purified by standard protocols.

In the RNA-based methods of the invention, the biomarker is preferably a SNP or a splice-variant.

Numerous methods are known in the art for the detection of biomarkers such as single-nucleotide polymorphisms (SNPs), splice-variants, haplotypes and microsatellites. The invention is not limited to any one detection method. These methods include nucleic acid sequencing; micro-sequencing; allele-specific oligonucleotide hybridization; size analysis, e.g., gel electrophoresis; hybridization; 5′ nuclease digestion; single-stranded conformation polymorphism; allele specific hybridization; primer specific extension; oligonucleotide ligation assay; restriction enzyme analysis; mass spectrophotometry and microarrays.

With regard to the detection of RNA, one or more of the above methods may be used (where appropriate) directly on the RNA. Alternatively, cDNA may be first obtained from the RNA.

Polymerase chain reaction (PCR) may or may not be used as part of the detection method of the invention. Using PCR, the presence or absence of the DNA biomarkers in or near the transferrin gene may be detected by amplifying a region of DNA in or near the transferrin gene using first and second nucleic acid primers. The primers will preferably have sequences and lengths so as to hybridize specifically under appropriate conditions to the appropriate region of the transferrin gene or near the transferrin gene. In some embodiments, the primers will not hybridize to the SNPs or microsatellites, but will flank these regions so that the SNPs or microsatellites will be included in the fragment that is amplified. Primers may be designed using a number of different software programs, locally on a computer or on numerous websites (for example the program “Primer3” at http://frodo.wi.mit.edu/), or alternatively, using the SNP analysis program of the WatCut package found at

http://watcut.uwaterloo.ca/watcut/watcut/template.php. The primers are preferably DNA primers.

The invention particularly relates therefore to a method as disclosed herein which additionally comprises the step of amplifying the region of the fish DNA which includes the biomarker(s) in question. Preferably, the biomarker is one or more of the SNPs or microsatellites given in Table 2 or 3, or the corresponding SNPs or microsatellites in the fish in question. Preferably, the amplifying is by an exponential amplification method, e.g. the polymerase chain reaction (PCR).

In a further aspect, the invention provides a kit comprising at least one nucleic acid primer, wherein the nucleic acid primer is capable of hybridising under high stringency conditions to the coding or non-coding strand of a region of DNA in or near the transferrin gene and which can be used in an exponential amplification reaction to amplify a region of DNA which comprises at least one of the microsatellites given in Table 2 or 3, or at least one of the SNPs in Table 2 or 3, or corresponding SNPs or microsatellites in the fish in question.

The length of the amplified DNA which includes the microsatellite will generally be a length which will allow for the detection of the presence or absence of the microsatellite by a suitable method and the detection of the allele variant by measuring the length of the PCR-product, usually with electrophoresis. The goal of the analysis is to identify the allele-length of the microsatellite of the given individual (if the individual is a homozygote for the microsatellite) or the two allele lengths if the individual is a heterozygote for the microsatellite. Preferably, the length of the region of amplified nucleic acid will be 50-2000 nucleotides, and more preferably 80-500 nucleotides.

The identification of the SNP-allele is also usually performed with methods including an initial PCR-reaction, but since the lengths of alternative SNP-variants is the same, other standard methods are used for SNP-typing. A number of such methods are available, from the use of restriction enzymes cutting the amplified DNA-strand (PCR-product) depending on the presence or absence of a specific nucleotide, sequencing, hybridisation techniques, mini-sequencing, mass spectrophotometry, etc. All these methods are standard and well known for the person skilled in the field.

Preferably, the primers used in the amplification process are between 15-50 nucleotides in length, more preferably 18-35 nucleotides and most preferably 20-25 nucleotides in length. The upstream and downstream primers might have different lengths.

In preferred embodiments, the primers are DNA primers whose nucleotide sequence consist of or comprise the nucleotide sequences given in SEQ ID NOs: 5, 6, 7 or 8.

In the present context, the term “high stringency conditions” refers to hybridization in 50% formamide, 1 M NaCl, 1% SDS at 37° C.; and a final wash in 0.1×SSC at 60 to 65° C. for 30 minutes. Optionally, wash buffers may comprise about 0.1% to 1% SDS. Duration of hybridization is generally less than about 24 hours, usually about 4 to 12 hours.

As indicated in Table 2 and Table 3, a number of the SNPs in the transferrin gene result in amino acid changes in the transferrin polypeptide. Consequently, an indication of the saltwater tolerance of the fish can be obtained by determining, directly or indirectly, which isoform of transferrin is present in the fish in question.

The invention therefore provides a method of obtaining an indication of the saltwater tolerance of a fish, the method comprising determining the presence in the fish of a transferrin polypeptide isoform which is associated with high saltwater tolerance.

The presence of a transferrin polypeptide isoform which is associated with high saltwater tolerance is indicative of high saltwater tolerance of the fish.

Preferably, the transferrin polypeptide isoform is one which is indicative of a high level of saltwater tolerance, i.e. it contains one or more of the SNPs selected from those listed in Haplotype 2 in Tables 2 or 3 or the SNPs at corresponding positions in the transferrin polypeptide in the fish in question. The presence of one or more of the aforementioned SNPs is indicative of high saltwater tolerance of the fish.

Preferably, the presence or absence of at least 2, 3, 4, 5, 10, 20 or all of the SNPs shown Table 2 or Table 3 is determined in the transferrin polypeptide of the fish or the SNPs at the corresponding positions in the transferrin polypeptide in the fish in question.

The invention further provides a method of obtaining an indication of the saltwater tolerance of a fish, the method comprising determining the presence in the fish of a transferrin polypeptide isoform which is correlated with low salt water tolerance.

The presence of a transferrin polypeptide isoform which is correlated with low salt water tolerance and the absence of a transferrin polypeptide isoform which is correlated with high salt water tolerance is indicative of low saltwater tolerance of the fish.

In other preferred embodiments, the transferrin polypeptide isoform is one which is indicative of a low level of saltwater tolerance, i.e., it contains one or more of the SNPs selected from those listed in Haplotype 1 in Tables 2 or 3 or the SNPs at corresponding positions in the transferrin polypeptide in the fish in question. The presence of one or more of the aforementioned SNPs is indicative of low saltwater tolerance of the fish.

The transferrin polypeptide may be obtained from any tissue of the fish which expresses transferrin. Furthermore, transferrin is transported around the fish body in plasma supplying most organs with iron, and hence plasma may also be used. As mentioned above, transferrin is primarily expressed in the liver, but it is also expressed in several other organs, depending on the fish species. In tilapia, transferrin is expressed in brain, gills and liver, inter alia. The transferrin polypeptide may be extracted and purified by standard protocols.

The transferrin isoforms may be distinguished by any suitable method, including immunological methods and protein sequencing methods. Examples of suitable methods include the fractionation of the polypeptides by one or two dimensional SDS-PAGE, optionally followed by Western blotting with an appropriate antibody; and various chromatographic methods including HPLC and protein identification using mass spectrometric (MS)-based methods (Rappsilber and Mann, 2002). Alternatively, the C-terminal sequence of a purified transferrin-containing fraction or fragments thereof may be sequenced. Alternatively, the protein truncation test (PTT) may be used (Roest, et al., (1993); van der Luijt, et al., (1994)).

The presence of amino acid differences in the transferrin polypeptides from saltwater tolerant and saltwater sensitive fish means that the transferrin polypeptides from these two types of fish can be distinguished by immunological means, for example, using antibodies.

The invention therefore provides a method of obtaining an indication of the saltwater tolerance of a fish, the method comprising:
(i) detecting whether a first antibody binds to a transferrin-containing sample obtained from the fish, wherein said first antibody is an antibody which binds specifically to a transferrin polypeptide which has one or more of the amino acid changes of Haplotype 2 at the positions shown in Table 2 or Table 3 or at corresponding positions in the transferrin polypeptide of the said fish, wherein the antibody does not bind to a transferrin polypeptide which does not have any of the amino acid changes of Haplotype 2 at the positions shown in Table 2 or Table 3 or at corresponding positions in the transferrin polypeptide of the said fish,
   and/or
(ii) detecting whether a second antibody binds to a transferrin-containing sample obtained from the fish, wherein said second antibody is an antibody which binds specifically to a transferrin polypeptide which does not have any of the amino acid changes of Haplotype 2 at the positions shown in Table 2 or Table 3 or at corresponding positions in the transferrin polypeptide of the said fish, wherein the second antibody does not bind to a transferrin polypeptide which has one or more of the amino acid changes of Haplotype 2 at the positions shown in Table 2 or Table 3 or at corresponding positions in the transferrin polypeptide of the said fish.

In the above method, if the first antibody binds to the transferrin in the sample, the fish is one with a high level of saltwater tolerance. If the second antibody also binds to the transferrin in the sample, then this is indicative of a fish being a heterozygote containing both the high and the low resistance-associated haplotype. The heterozygotes would have an improved saltwater tolerance compared to individuals homozygous for the low tolerance haplotype.

If only the second antibody binds to the transferrin-containing sample, this is indicative of the fish having a low level of saltwater tolerance.

The invention also provides an antibody which binds specifically to a transferrin polypeptide which has one or more of the amino acid changes of Haplotype 2 at the positions shown in Table 2 or Table 3 or at corresponding positions in the transferrin polypeptide of the said fish, wherein the antibody does not bind to a transferrin polypeptide which does not have any of the amino acid changes of Haplotype 2 at the positions shown in Table 2 or Table 3 or at corresponding positions in the transferrin polypeptide of the said fish.

The invention also provides an antibody which binds specifically to a transferrin polypeptide which does not have any of the amino acid changes of Haplotype 2 at the positions shown in Table 2 or Table 3 or at corresponding positions in the transferrin polypeptide of the said fish, wherein the antibody does not bind to a transferrin polypeptide which has one or more of the amino acid changes of Haplotype 2 at the positions shown in Table 2 or Table 3 or at corresponding positions in the transferrin polypeptide of the said fish.

The invention also provides a kit comprising:
(i) an antibody which binds specifically to a transferrin polypeptide which has one or more of the amino acid changes of Haplotype 2 at the positions shown in Table 2 or Table 3, wherein the antibody does not bind to a transferrin polypeptide which does not have any of the amino acid changes of Haplotype 2 at the positions shown in Table 2 or Table 3,
   and/or
(ii) an antibody which binds specifically to a transferrin polypeptide which does not have any of the amino acid changes of Haplotype 2 at the positions shown in Table 2 or Table 3, wherein the antibody does not bind to a transferrin polypeptide which has one or more of the amino acid changes of Haplotype 2 at the positions shown in Table 2 or Table 3.

Optionally, the kit also contains appropriate instructions for use. Preferably, the antibody is one which specifically binds to a polypeptide whose amino acid sequence consists of the sequence given in SEQ ID NO: 2, wherein the antibody does not bind to a polypeptide whose amino acid sequence consists of the sequence given in SEQ ID NO: 15.

In another preferred embodiment, the invention provides an antibody which binds to a polypeptide whose amino acid sequence consists of the sequence given in SEQ ID NO: 15, but which does not bind to a polypeptide whose amino acid sequence consists of the sequence given in SEQ ID NO: 2.

Subtraction methods for the isolation of such antibodies are well known in the art.

The antibodies may be of any suitable source, e.g., monoclonal, polyclonal, chimeric, bispecific, single-chain or fragments thereof. Antibody fragments include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals, or derived from phage or ribosome display libraries. Preferably, the antibodies are human, murine (e.g., mouse or rat), donkey, rabbit, goat, guinea pig, camel, horse, or chicken. The antibodies of the invention may be monospecific, bispecific, trispecific or of greater multispecificity.

The antibodies may be labelled in any suitable manner, thus allowing for detection in a suitable assay.

The invention as described herein discloses for the first time the correlation between saltwater tolerance and biomarkers in the transferrin gene. A number of microsatellites, SNPs and haplotypes are disclosed herein as evidence of this correlation. The invention also provides methods of identifying new biomarkers. Typing some or all of the set of microsatellites and SNPs of the invention can be used to identify new haplotypes consisting of any combination of the single alleles in each position. The typing of the two microsatellites, for example, using the sequence information described herein, can be used to identify new alleles of different lengths, compared to the ones identified in the studied fish. Such alleles could identify new haplotypes, containing other transferrin variants, and these can be associated with a different saltwater resistance. Every single SNP described herein can also be typed, and new haplotypes detected comprising new combinations of SNPs, each of which could be associated with a specific functional effect and/or a specific saltwater tolerance.

In a yet further embodiment therefore, the invention provides a method of finding a biomarker which is indicative of saltwater tolerance in a fish the method comprising determining the presence in or near the transferrin gene of a biomarker which is present in fish which are saltwater tolerant but is not present in fish which are not saltwater tolerant.

The biomarker is preferably one or more SNPs, a haplotype or a microsatellite. Differential expression studies can be used in the above methods.

It is also possible to perform testing of fish early, to avoid performing practical saltwater trials. It is valuable to use the information about transferrin genotypes/haplotypes in experiments to find new genes involved in saltwater resistance. By genetic typing, the genotypes/haplotypes can be used in the design of projects to standardize transferrin-variants of fish included in the studies. Such design will reduce confounding or disturbing genetic background effects and potential gene interactions, and it will be easier to detect other genes or chromosomal regions involved in salt-water tolerance. Alternatively, transferrin-haplotyping can be corrected for statistically in the analysis phase of new projects, to control for the effect of transferrin.

The method of the invention can also be used to identify biomarkers in other genes (i.e., genes other than transferring.

The invention therefore provides a method of finding a biomarker other than transferrin which is indicative of saltwater tolerance in a fish, the method comprising:
(i) providing a population of fish which have a first biomarker in or near the transferrin gene of the fish, wherein the first biomarker is one which is correlated with saltwater tolerance of the fish,
(ii) establishing the presence in a DNA, RNA or polypeptide sample of the fish of a second biomarker which is correlated with the presence of the first biomarker, wherein the second biomarker is not in or near the transferrin gene, in the transferrin RNA or in the transferrin polypeptide wherein the second biomarker is one which is indicative of salt tolerance of the fish.

Preferably, the correlation between the presence of the first and second biomarkers is a significant correlation, e.g., p<0.05, 2-tailed test.

In a yet further embodiment, the invention provides a method of producing a fish, the method comprising the steps:
(i) selecting a first fish whose saltwater tolerance has been determined by a method of the invention,
(ii) breeding said first fish with a fish of the opposite sex in order to produce progeny fish.

The invention also provides a method of producing a fish, the method comprising the steps:
(i) determining the saltwater tolerance of a first fish by a method of the invention,
(ii) breeding said first fish with a fish of the opposite sex in order to produce progeny fish.

In the above context, the term “breeding” includes artificial methods of breeding, including the isolation of male and/or female gametes and the in vitro or in vivo combing of such gametes.

Optionally, the second fish is also one whose saltwater tolerance has been determined by a method of the invention.

The invention therefore particularly provides a method of producing a fish, the method comprising the steps:
(i) selecting a first fish whose saltwater tolerance has been determined by a method of the invention,
(ii) obtaining gametes from the first fish,
(iii) combining the gametes from the first fish with gametes from a second fish of the opposite sex in order to produce progeny fish.

The invention also provides a method of producing a fish, the method comprising the steps:
(i) determining the saltwater tolerance of a first fish by a method of the invention,
(ii) obtaining gametes from the first fish,
(iii) combining the gametes from the first fish with gametes from a second fish of the opposite sex in order to produce progeny fish.

The invention also relates to products produced from the fish of the invention, e.g., cuts of fish.

The invention also provides a method of producing progeny fish, comprising the steps:
(i) determining the saltwater tolerance of a population of fish by a method of the invention;
(ii) selecting two fish from the population, wherein the two fish are of opposite sex and both have high saltwater tolerance;
(iii) breeding the two fish of opposite sex in order to produce progeny fish; and
(iv) selecting progeny which have high saltwater tolerance.

Preferably, the progeny fish which are selected are those which are homozygous for the high saltwater tolerance genotype, e.g., wherein the progeny fish are homozygous for Haplotype 2 as shown in Table 2 or Table 3 or the corresponding Haplotype in the fish in question.

In a yet further embodiment, the invention provides a method for producing a transgenic fish, the method comprising integrating a transferrin gene stably into the genome of the fish in a position such that the transferrin gene is expressed in some or all tissues of the fish.

A further aspect of the invention provides a transgenic fish, wherein the fish comprises a heterologous transferrin gene stably-integrated into its genome in a position such that the transferrin gene is expressed in some or all tissues of the fish. The transferrin gene may have a nucleotide sequence which is the same as that of the endogenous transferrin gene in the fish in question or it may have a different nucleotide sequence, for example, the sequence of a transferrin gene from another fish species or other animal species (e.g., a mammalian transferrin gene). Preferably, the transferrin gene is a heterologous or foreign transferrin gene, i.e., it has a nucleotide sequence which is different from the endogenous transferrin gene in the fish in question.

Preferably, the transferrin gene has a nucleotide sequence which corresponds to the nucleotide sequence of a transferrin gene which is obtainable from a fish with high saltwater tolerance.

In a further embodiment, a particularly preferred sequence is that comprising SEQ ID NO: 14 or a nucleotide sequence which encodes the amino acid sequence given in SEQ ID NO: 15.

The invention also provides a method of increasing the saltwater tolerance of a fish, comprising introducing into the fish an agent which increases the transferrin levels in the fish.

Preferably, the agent is one which increases the level of a transferrin isoform which has been shown to be associated with high saltwater tolerance, e.g., a transferrin isoform consisting of or comprising the amino acid sequence given in SEQ ID NO: 15 or the corresponding transferrin isoform in the fish in question.

In some embodiments of the invention, the agent is transferrin. Preferably, the transferrin has been obtained from fish with a high salt water tolerance.

In other embodiments, the agent is one which indirectly stimulates the production of transferrin in the fish.

The agent may be introduced into the fish in any suitable means, for example, as part of the fish's food or in the water.

The invention also provides fish produced by the above methods, as well as germ cells, e.g., eggs and sperm, from the aforementioned fish.

The invention also provides a method of producing fish sperm, comprising the steps:
(i) determining the saltwater tolerance of a population of fish by a method of the invention;
(ii) selecting a male fish from the population; and
(iii) obtaining sperm from the male fish.

Preferably, the obtained sperm is then stored, most preferably under refrigerated conditions. The invention also relates to sperm obtained by this method.

The invention also provides a method of producing fish eggs, comprising the steps:
(i) determining the saltwater tolerance of a population of fish by a method of the invention;
(ii) selecting a female fish from the population; and
(iii) obtaining eggs from the female fish.

Preferably, the obtained eggs are then stored, most preferably under refrigerated conditions. The invention also relates to eggs obtained by this method.

The invention also provides a nucleic acid molecule whose nucleotide sequence comprises the sequence shown in SEQ ID NO: 14 or which encodes the amino acid sequence shown in SEQ ID NO: 15.

The invention also provides a vector or plasmid wherein the nucleotide sequence of the vector or plasmid comprises the nucleotide sequence shown in SEQ ID NO: 14; and a host cell comprising the aforementioned vector or plasmid. The host cell may be a prokaryotic or eukaryotic host cell, for example, a bacterial, fungal, animal or plant host cell.

Furthermore, the invention provides a polypeptide wherein the amino acid sequence of the polypeptide consists of or comprises the amino acid sequence shown in SEQ ID NO: 15.

The invention will be further described by the following non-limiting examples. Those of skill will readily recognize a variety of noncritical parameters which could be changed or modified to yield essentially similar results.

### EXAMPLES

### Example 1

### Biological Material

A saltwater tolerance experiment was performed on a Nile tilapia population. Salt concentration was gradually increased from 0-30 ppt over three days in the containers keeping full-sibs from five different families. We collected 23 surviving offspring at the final salt concentration to compare with 24 non-survivors from one family (family no. 1) in addition to 37 survivors and 40 non-survivors from a second family (family no. 2). Fin clips were stored in 96% ethanol.

DNA was extracted with the MagAttract DNA M48 kit (Qiagen) as recommended. Fin fragments were dissolved in lysis buffer and proteinase K over night at 37° C. The automated isolation was completed on a BioRobot M96 Workstation (Qiagen) and DNA was eluted by volumes of 50 μl. Total-RNA from Nile tilapia gills and brains, preserved in RNAlater® (Ambion) at −20° C., was isolated with RNeasy Midi Kit (Qiagen) after standard protocol. The organs were added the appropriate amount of RLT-buffer (lysis) and homogenized by a roller in sterile plastic bags (Seaward, Stomacher) and additional homogenizing was carried out by passing the lysate through a sterile 19G syringe. RNA was eluted twice with 150 μl RNase-free water and precipitated with ethanol after standard procedure to increase the concentrations. The concentrations were measured on a Lambda EZ 201 (Perkin Elmer) and the organs pooled. The total RNA was then treated with DNA-free™ (Ambion), as recommended by the protocol, to remove contaminating DNA from the extracted RNA.

### Example 2

### Sequencing of Transferrin

Based on published transferrin sequences of the tilapia species O. aureus and O. mossambicus of exon 7, 9 and 10 (acc. no. AJ318861 and AJ312311), primers were constructed for forward and reverse gene-walking. Gene-walking was performed using the DNA Walking SpeedUp™ Kit (Seegene, Inc.). The gene-walking process consists of several steps of PCRs using a set of universal PCR primers provided with the kit in combination with our TF-specific designed PCR-primers in both directions.

The temperature profile and reaction setup were followed as recommended in the protocol. The products were tested on 1% agarose gel and purified with ExoSAP-IT before the selected samples were sequenced after standard protocol with BigDye® Terminator (v3.1) Cycle Sequencing Kit (Applied Biosystems) with 0.25 μl 10 μM Universal primer (Seegene, Inc.) and 0.5 μl 5 μM of the respectively nested and 2.nested designed primers, on an ABI PRISM® 3100 Genetic Analyzer (Applied Biosystems).

As a supplement to the gene-walking we used RACE-technology and RT-PCR on total-RNA to amplify cDNA sequence data.

The RACE-technology was carried out as recommended by the GeneRacer™ RACE Ready cDNA Kit Manual (Invitrogen). Gene specific primers (GSP), forward, reverse and nested, were constructed based on existing sequence, based on the protocol's recommendations. All steps were performed as recommended by the protocol. The PCR-products were purified by ExoSAP-IT (Amersham Biosciences) and then sequenced after standard protocol with BigDye® Terminator (v3.1) Cycle Sequencing Kit (Applied Biosystems) and the respective PCR primers, reverse and forward, on an ABI PRISM® 3100 Genetic Analyzer (Applied Biosystems).

The two-step protocol of Ready-to-Go-RT-PCR Beads (Amersham Biosciences) was performed as recommended. The PCR-products were purified by ExoSAP-IT (Amersham Biosciences) and then sequenced as described above.

New primers were constructed to run PCR across the introns, based on the obtained exon sequences. PCR conditions were as follows: 2 μl 10×PCR buffer including 15 mM MgCl₂ (Applied Biosystems), 2 μl dNTP (2 mM), 0.5 μl MgCl₂ (25 mM), ˜75 ng DNA, 1 μl PCR primer, forward and reverse (5 pmol/μl), 0.2 μl AmpliTaq® DNA Polymerase (5 U/μl, Applied Biosystems) and water to a total volume of 20 μl. Annealing temperature was 58° C. for 1 minute and the PCR was performed with 35 cycles. The PCR-products were sequenced by the method described above. Then DNA and cDNA sequences were aligned by Sequencher 4.1.4 (Gene Codes Co.) to reveal the exon-intron boundaries. Alignments were also performed by blastn (Basic Local Alignment Search Tool for nucleotides) and by bl2seq (aligning of two sequences) at NCBI. The transferrin sequence of Japanese medaka (Oryzias latipes) (acc. no.: D64033) was used as reference sequence.

The complete DNA sequence of the revealed transferrin gene in Nile tilapia, with detailed information of exon and intron boundaries, will also be available in GenBank (acc. no.: DQ272465) after filing of the patent application. The gene consists of 7010 base pairs, with 17 exons separated by 16 introns, including parts of the 5′ and 3′UTR regions. (SEQ ID NO:1) The complete coding domain sequence (CDS) is composed by 2085 bp (SEQ ID NO:2).

The Nile tilapia transferrin protein consists of 694 amino acids. A protein-protein BLAST (blastp) gave highest similarity match with the transferrin protein sequence of medaka with identity score of 77% followed by red and black sea bream and brown trout with 73, 72 and 67% identity, respectively. A detailed overview of this protein comparison is given in FIG. 1 which shows several highly conserved regions and some regions with diverging amino acids among all species. Also observed is an insertion of three amino acids in the protein sequence of tilapia only.

The iron and anion binding residues in the two lobes of the protein is found conserved as described by (Lambert et al., 2005) in the five fish species compared, except for one residue where tilapia has an aspartic acid instead of the conserved histidine in the N-lobe of the protein.

### Example 3

### Genotyping of Microsatellites Closely Linked to Transferrin

Microsatellites linked to transferrin were identified by screening an available tilapia pooled BAC-library (Katagiri et al., 2001) for a BAC clone containing the gene, by PCR amplification with primers designed from available sequence of the gene (Cnaani et al., 2002). A Clone BAC DNA kit (Princeton Separations) was used to isolate the BAC clone following the recommended protocol. The clone was then fragmented by a suitable restriction enzyme and the DNA fragments, of 500-1000 bp, were purified by a StrataPrep® Gel Extraction Kit (Stratagene).

The BAC DNA fragments were ligated to pUC19 Plasmid DNA vectors (Sigma-Aldrich Co) and transformed into XL10-Gold® Ultracompetent Cells (Stratagene) following the given instructions. Hybridization techniques were carried out by traditional methods (Sambrook, 1989) using Colony/Plaque Screen™ nylon membranes (NEF 990A, Perkin Elmer NEN) for colony-lift. gt₁₀ and ct₁₀ -probes were end-labeled at 37° C. for 40 min, 95° C. for 10 min and 15 min on ice, in a solution of 4 μl gt₁₀-probe (1 μg/μl, MGW), 4 μl ct₁₀-probe (1 μg/μl, MGW), 6 μl T4 Polynucleotide Kinase 10 U/μl (NEB), 12 μl T4 Polynucleotide Kinase Reaction Buffer 10× (NEB), 50 μl [γ³²P] ATP 500 μCi, (Amersham Biosciences) and 44 μl H₂O.

Labeled probes were added to the pre-hybridization solution, and the filters were hybridized overnight in accordance with standard methods. The filters were washed and then covered in Saran Wrap and exposed to Hyperfilm MP (Amersham Biosciences) overnight at −70° C. before the films were developed. Positive colonies were identified and a secondary screening was performed as described for the first screening steps.

The positive clones were picked and DNA plasmids were purified with QIAprep Spin Miniprep Kit (QIAGEN) as recommend by the protocol. The vector inserts were sequenced to find the potential microsatellites, after the standard protocol for the BigDye® Terminator v3.1 Cycle Sequencing Kit (Applied Biosystems), with M13 primers, on ABI PRISM® 3100 Genetic Analyzer (Applied Biosystems).

To verify that the picked BAC clone included the correct gene the isolated BAC-clone was amplified by PCR with the same primer pair used for finding the specific clone in the BAC library. The PCR product was then sequenced as described above, now with the respective PCR primers. The results were subsequently run by BLAST and that way verifying that the sequence matched the correct gene (Rengmark et al., 2006).

Two microsatellite markers were identified in the BAC-clone containing the transferrin gene. TF-A consisted of an repeated sequence of (GT)₁₀ (SEQ ID NO:3) and TF-B of repeated sequence of (GT)₁₄ (SEQ ID NO:4).

The two microsatellites closely linked to transferrin, TF-A and TF-B, were genotyped on the surviving and non-surviving tilapias from family 1 and 2 in the saltwater tolerance experiment. PCR conditions were as reported above, but with an annealing temperature of 55° C. for 45 seconds and 32 cycles. The PCR primers used to genotype marker TF-A were SEQ ID NO:5 and 6 and for marker TF-B: SEQ ID NO:7 and 8. The samples were run on an ABI PRISM® 3100 and the results were analyzed by GeneMapper v 3.0 (Applied Biosystems).

### Haplotypes of Salt Tolerant Tilapia

The two loci were inherited as one haplotype block. Haplotype 1 consisted of the alleles 287 and 184 and haplotype 2 of the alleles 289 and 188, represented by marker TF-A and TF-B respectively. The parents tested were heterozygous for both markers, except the sire in family 2, which was homozygous for both loci with the respective alleles 287 and 184. The haplotypes and χ₂-distribution are presented in Table 1. We observed that salt tolerant fish showed a tendency of possessing haplotype 2 and the less salt tolerant fish had a majority of haplotype 1. The segregation distortion was significant at p<0.025 and 0.05.

TABLE 1
χ₂-distribution of the two haplotypes in two QTL-families
shows that salt tolerant individuals possess haplotype 2.

| | QTL-family 1 * | | | | QTL-family 2 ** | | |
|---|---|---|---|---|---|---|---|
| | Salt | Early | | | Salt | Early | |
| | tolerant | dead | Total | | tolerant | dead | Total |
| Haplotype 2 | 26 | 15 | 41 | Haplotype 2 | 24 | 14 | 38 |
| Haplotype 1 | 20 | 33 | 53 | Haplotype 1 | 50 | 66 | 116 |
| Total | 46 | 48 | 94 | Total | 74 | 80 | 154 |
| Individuals | 23 | 24 | | Individuals | 37 | 40 | |

| | |
|---|---|
| Degrees of freedom: 1 | Degrees of freedom: 1 |
| Chi-square = 6.10 | Chi-square = 4.61 |
| p is less than or equal to 0.025 | p is less than or equal to 0.05 |
| The distribution is significant | The distribution is significant |

* Family 1: Both parents with heterozygous haplotype 1/2
** Family 2: One parent with heterozygous haplotype 1/2, the other with homozygous haplotype 1/1

### Example 4

### Identifying SNPs in the Transferrin Gene

Additional primer pairs were designed to find potential SNPs in the expressed part of the gene. Standard PCRs were first performed on the family 2-parents, then sequenced by the MegaBACE™ 1000 DNA Analysis Systems (Amersham Biosciences) using the DYEnamic™ ET Dye Terminator Kit (Amersham Biosciences). Reaction conditions were as follows: 4 μl ET reagent premix, 4.5 μl H₂O, 1 μl PCR-product and 0.5pl primer (5 μM) with the following step repeated 28 times: 95° C., 15 seconds, 58° C., 10 seconds, 60° C., 1 minute. The post reaction clean-up was performed as recommended by the protocol with ethanol and 7.5M ammonium acetate. SNPs were identified by aligning and comparing the sequence data by Sequencher 4.1.4 (Gene Codes Co.). If SNPs were detected, the two offspring groups in the family (salt-water survivors and non-survivors) were then sequenced over the determined SNPs to define their genotypes.

### SNPs Detected in the Transferrin Gene

25 heterozygous SNPs were identified in the transferrin gene, 15 of these were located in exon 7, 8, 14, 15 and 16. Fourteen of these resulted in an amino acid change (Table 2).

We observed that all the SNPs, genotyped on offspring from family 2, segregated as two haplotype blocks that also include the two microsatellites. The haplotype distribution is shown in detail in Table 1 and the linkage distortion strongly implies that the individuals possessing haplotype 2 are more salt tolerant. A complete overview of the haplotypes is given in Table 3.

The iron and anion binding residues are all conserved except for one (FIG. 1). The histidine residue in the N-lobe codes for an aspartic acid (GAC) in Nile tilapia (position 258). All the other fish species have His in this position (CAC or CAT). Lambert et al. (2005) reports that this His-position is the most variant residue in the N-lobe and Asp in this position is also found in different insects like fruit fly and termite. Interestingly, this position in exon 7 contains a SNP (see Table 2) in the neighbouring amino acid just upstream for the Asp resulting in an amino acid change from alanine to glycine in the surviving group of fish.

TABLE 2
SNPs detected in the transferrin gene.

| | Gene | | | | Amino acid | Amino | No. in |
|---|---|---|---|---|---|---|---|
| | position: | Bp no.: | Haplotype 1: | Haplotype 2: | change: | acid no.: | FIG. 1 |
| 1 | ex7 | 2968 | C | G | Ala (A) → Gly (G) | 256 | 257 |
| 2 | ex8-1 | 3190 | G | A | Ala (A) → Thr (T) | 295 | 296 |
| 3 | ex8-2 | 3224 | G | A | Arg (R) → Lys (K) | 306 | 307 |
| 4 | ex8-3 | 3229 | T | G | Leu (L) → Val (V) | 308 | 309 |
| 5 | intr8 | 3330 | A | G | — | — | — |
| 6 | ex14 | 5368 | G | A | Val (V) → Ile (I) | 545 | 546 |
| 7 | intr14-1 | 5437 | G | A | — | — | — |
| 8-9 | intr14-2 | 5462-3 | GT | CA | — | — | — |
| 10 | intr14-3 | 5467 | C | T | — | — | — |
| 11 | ex15-1 | 5521 | G | T | no change | 567 | 568 |
| 12 | ex15-2 | 5598 | C | T | Ala (A) → Val (V) | 593 | 594 |
| 13 | intr15-1 | 5689 | G | T | — | — | — |
| 14 | intr15-2 | 6530 | C | T | — | — | — |
| 15 | ex16-1 | 6549 | C | T | Ala (A) → Thr (T) | 617 | 618 |
| 16-18 | ex16-2 | 6562-4 | GCT | AGC | Ala (A) → Ser (S) | 622 | 623 |
| 19 | ex16-3 | 6659 | A | G | Glu (E) → Gly (G) | 654 | 655 |
| 20-23 | ex16-4 | 6685-8 | GAAG | ATTT | Glu (E) Ala (A) → | 663-4 | 664-5 |
| | | | | | Ile (I) Ser (S) | | |
| 24-26 | ex16-5 | 6697-9 | GAT | ACC | Asp (D) → Thr (T) | 667 | 668 |
| 27 | ex16-6 | 6729 | T | A | Asp (D) → Glu (E) | 677 | 678 |
| 28 | ex16-7 | 6733 | G | A | Ala (A) → Thr (T) | 679 | 680 |
| 29 | ex16-8 | 6736 | T | C | Ser (S) → Pro (P) | 680 | 681 |
| 30 | intr16-1 | 6769 | T | C | — | — | — |
| 31 | intr16-2 | 6777 | A | T | — | — | — |
| 32 | intr16-3 | 6780 | A | C | — | — | — |
| 33 | intr16-4 | 6785 | A | G | — | — | — |
| 34 | intr16-5 | 6848 | C | A | — | — | — |

Listed are location in gene, base pair number (correspond with sequence in GenBank, acc. no.; DQ272465), genotype in wild type and SNP, amino acid change if any and the amino acid position (see also FIG. 1 where these positions are underscored).

TABLE 3
Overview of transferrin haplotypes 1 and 2 across the two genetically-linked
microsatellite markers (TF-A and TF-B) and the 34 SNPs (within
the gene) in salt-tolerant Nile tilapia.

| | TF-A | TF-B | ex7 | ex8 | | | intr8 | ex14 | intr14 | | | ex15 | | intr15 | |
|---|---|---|---|---|---|---|---|---|---|---|---|---|---|---|---|
| 1 | 287 | 184 | C | G | G | T | A | G | G | GT | C | G | C | G | C |
| 2 | 289 | 188 | G | A | A | G | G | A | A | CA | T | T | T | T | T |

| | ex16 | | | | | | | | intr16 | | | | |
|---|---|---|---|---|---|---|---|---|---|---|---|---|---|
| 1 | C | GCT | A | GAAG | GAT | T | G | T | T | A | A | A | C |
| 2 | T | AGC | G | ATTT | ACC | A | A | C | C | T | C | G | A |

### Example 5

### Expression Study on Transferrin

A parallel saltwater experiment was carried out for the expression study. A total of 200 Nile tilapias of four different families were used in this experiment. The average body length was 10 cm and there was an approximately equal distribution of the sexes. Twenty-five fish from each family were pooled in two tanks with identical physical environments. The salt concentration in one of the tanks was gradually increased every second day from 0-32 ppt during a period of ten days, and the fish were kept for another five days in this saline condition. All the fish were then killed and dissected. Brains and gills were directly transferred to and preserved in RNAlater® (Ambion) at −20° C.

Brains and gills from each treatment were pooled in separate containers and total-RNA was extracted as recommended by the RNeasy Maxi Kit (Qiagen). Equal amounts of the brain and gill isolations were mixed together to give a salt and freshwater pool respectively. The intention of organ pooling was to reveal a total expression of the salt tolerance metabolism.

Transferrin was analyzed by RT-PCR on an ABI PRISM® 7700 Sequence Detection System (Applied Biosystems). Total-RNA was treated with DNA-free™ (Ambion) to remove contaminating DNA, as recommended by the protocol. A two-step RT-PCR was performed with a TaqMan® Gold RT-PCR Kit (Applied Biosystems) with PCR reagents and cycling conditions as recommended. This two-step RT-PCR includes the addition of AmpErase UNG, which can prevent carryover contamination from PCR products. Specific TaqMan® primers and probes were constructed in terms of the protocol. Several incremental dilutions were done in advance to test the best MgCl₂, primer and probe concentrations for optimal RT-PCR conditions. Standard deviations for each sample were measured based on three parallels runs.

Successful RT-PCRs were run on pools of total-RNA from tilapia brain and gills, in addition to gills only. This states that transferrin is expressed in gills and indicates expression also in brain, which up to this point has not been detected in other vertebrates than Atlantic cod (Denovan-Wright et al., 1996). Transferrin showed a significant up-regulation when analyzed by RT-PCR.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

### REFERENCES

Avella, M., Berhaut, J., Bornancin, M., 1993. Journal of Fish Biology 42, 243-254.
Bentsen, H. B., Eknath, A. E., Palada-de Vera, M. S., Danting, J. C., Bolivar, H. L., Reyes, R. A., Dionisio, E. E., Longalong, F. M., Circa, A. V., Tayamen, M. M., Gjerde, B., 1998. Aquaculture 160, 145-173.
Briggs, D. A., Sharp, D. J., Miller, D., Gosden, R. G., 1999. Molecular human reproduction 5, 1107-1114.
Cataldi, E., Crosetti, D., Conte, G., D'Ovidio, D., Cataudella, S., 1988. Journal of Fish Biology 32, 191-196.
Cnaani, A., Ron, M., Lee, B. Y., Hulata, G., Kocher, T. D., Seroussi, E., 2002. Mapping the transferrin gene in tilapia. Animal Genetics 33, 78-80.
Denovan-Wright, E. M., Ramsey, N. B., McCormick, C. J., Lazier, C. B., Wright, J. M., 1996. Part B, Biochemistry & molecular biology 113, 269-273.
Doize, F., Roux, I., Martineau-Doize, B., DeRoth, L., 1990. Can J Vet. Res. 54, 397-399.
Doudet, T., 1992. Aquaculture 102, 275-288.
Katagiri, T., Asakawa, S., Minagawa, S., Shimizu, N., Hirono, I., Aoki, T., 2001. Animal Genetics 32, 200-204.
Kocher, T. D., 2002. Tilapia Genomics and Applications. Available at http://nps.ars.usda.gov/static/arsoibiotecws2001/contributions/Kocher.htm.
Lambert, L. A., Perri, H., Halbrooks, P. J., Mason, A. B., 2005. Part B, Biochemistry & molecular biology 142, 129-141.
Maxam and Gilbert (Proc. Natl Acad Sci USA (1977) 74:560)
Park, I., Schaeffer, E., Sidoli, A., Baralle, F. E., Cohen, G. N., Zakin, M. M., 1985. Proceedings of the National Academy of Sciences of the United States of America 82, 3149-3153.
Rappsilber and Mann, Trends Biochem Sci. (2002) February; 27(2):74-8.
Rengmark, A. H., Slettan, A., Lee, W. J., Lie, Ø., Lingaas, F. 2006. Identification and mapping of genes associated with salt tolerance of tilapia. (UnPub)
Roderick, E., 1999. Seafood International 46-50.
Roest, et. al., (1993) Hum. Mol. Genet. 2:1719-21;
Sambrook, J., 1989. Molecular cloning: a laboratory manual. Cold Spring Harbor Laboratory, New York.
Sanger et al. (1977) Proc. Nat. Acad. Sci 74:5463.
Stafford, J. L., Belosevic, M., 2003. Developmental and comparative immunology 27, 539-554.
Trewavas, E., 1983. Tilapiine fishes: of the genera Sarotherodon, Oreochromis and Danakilia/Ethelwynn Trewavas. British Museum (Natural History), London.
Van der Luijt, et. al., (1994) Genomics 20:1-4
Yi, Y., Lin, C. K., Diana, J. S., 2002. Semi-Intensive Culture of Red Tilapia in Brackishwater Ponds. In: K. McElwee, K. Lewis, M. Nidiffer, and P. Buitrago (Editors), Nineteenth Annual Technical Report. Pond Dynamics/Aquaculture CRSP, Oregon State University, Corvallis, Oreg., 89-96.

We claim:

1. A method of determining the saltwater tolerance of a fish, the method comprising determining the status of a biomarker in a fish, wherein the biomarker is one which is correlated with high or low saltwater tolerance of the fish, and wherein the presence or absence of said biomarker is indicative of the saltwater tolerance of the fish.

2. The method of claim 1, wherein the biomarker is transferrin DNA, RNA or polypeptide, the glycosylation pattern of the transferrin polypeptide, or the iron-binding ability of the transferrin polypeptide.

3. The method of claim 2, wherein the biomarker is a variation of the nucleotide sequence of the DNA in or near the transferrin gene, a variation of the nucleotide sequence of the transferrin RNA, a variation of the amino acid sequence of the transferrin polypeptide.

4. The method of claim 3, wherein the biomarker is one or more single nucleotide polymorphisms (SNP), a haplotype, a microsatellite, an alternatively-spliced RNA product, or a polypeptide epitope.

5. The method of claim 4, wherein the biomarker is one or more single nucleotide polymorphism (SNP), and wherein the one or more SNP is at a position in the transferrin gene selected from the group consisting of: ex7 (bp2968), ex8-1 (bp3190), ex8-2 (bp3224), ex8-3 (bp3229), intr8 (bp3330), ex14 (bp5368), intr14-1 (bp5437), intr14-2 (bp5462-3), intr14-3 (bp5467), ex15-1 (bp5521), ex15-2 (bp5598), intr15-1 (bp5689), intr15-2 (bp6530), ex16-1 (bp6549), ex16-2 (bp6562-4), ex16-3 (bp6659), ex16-4 (bp6685-8), ex16-5 (bp6697-9), ex16-6 (bp6729), ex16-7 (bp6733), ex16-8 (bp6736), intr16-1 (bp6769), intr16-2 (bp6777), intr16-3 (bp6780), intr16-4 (bp6785), intr16-5 (bp6848).

6. The method of claim 3, wherein a DNA, RNA or polypeptide sample is obtained from the fish, wherein the biomarker is correlated with high saltwater tolerance of the fish, and wherein the presence of said biomarker in the sample is indicative of high saltwater tolerance of the fish.

7. The method of claim 6, wherein the DNA in the sample carrying the variation of the nucleotide sequence in or near the transferrin gene is amplified.

8. The method of claim 1 further comprising:
(a) obtaining a sample from the fish comprising transferrin polypeptide,
(b) contacting the sample with an antibody that specifically binds to a transferrin polypeptide comprising one or more of the amino acid changes of haplotype 2, selected from the group consisting of Ala256 (A)→Gly256 (G), Ala295 (A)→Thr295 (T), Arg306 (R)→Lys306 (K), Leu308 (L)→Val308 (V), Val545 (V)→Ile545 (I), Ala593 (A)→Val593 (V), Ala617 (A)→Thr617 (T), Ala622 (A)→Ser622 (S), Glu654 (E)→Gly654 (G), Glu663 (E) Ala664 (A)→Ile663 (I) Ser664 (S), Asp667 (D)→Thr667 (T), Asp677 (D)→Glu677 (E), Ala679 (A)→Thr679 (T), and Ser680 (S)→Pro680 (P),
(c) detecting the binding of the antibody,
wherein binding of the antibody indicates the saltwater tolerance of the fish.

9. A method for increasing the saltwater tolerance of a fish, the method comprising integrating a transferrin gene stably into the genome of the fish in a position such that the transferrin gene is expressed in some or all tissues of the fish, wherein expression of transferrin increases the saltwater tolerance of the fish.

10. The method of claim 9, further comprising:
(a) selecting a male fish from the population; and
(b) obtaining sperm from the male fish.

11. The method of claim 9, further comprising:
(a) selecting a female fish from the population; and
(b) obtaining eggs from the female fish.

12. The method of claim 9, further comprising:
breeding the saltwater tolerant fish of claim 8 with a fish of the opposite sex in order to produce progeny fish.

13. The method of claim 9, further comprising:
(a) obtaining gametes from the saltwater tolerant fish of claim 8,
(b) combining the gametes from the saltwater tolerant fish with gametes from a second fish of the opposite sex in order to produce progeny fish
(c) selecting progeny which have high saltwater tolerance.

14. The method of claim 9, wherein the fish is a teleost or bony fish.

15. The method of claim 9, wherein the fish is a Cichlidae, Salmonidae, Cyprinidae or Gadidae.

16. An isolated antibody, wherein the antibody specifically binds to a transferrin polypeptide comprising one or more of the amino acid changes of haplotype 2, selected from the group consisting of Ala256 (A)→Gly256 (G), Ala295 (A)→Thr295 (T), Arg306 (R)→Lys306 (K), Leu308 (L)→Val308 (V), Val545 (V)→Ile545 (I), Ala593 (A)→Val593 (V), Ala617 (A)→Thr617 (T), Ala622 (A)→Ser622 (S), Glu654 (E)→Gly654 (G), Glu663 (E) Ala664 (A)→Ile663 (I) Ser664 (S), Asp667 (D)→Thr667 (T), Asp677 (D)→Glu677 (E), Ala679 (A)→Thr679 (T), and Ser680 (S)→Pro680 (P).

17. An isolated polypeptide comprising the amino acid sequence of SEQ ID NO: 15.

18. An isolated nucleic acid molecule encoding the polypeptide of claim 17 or comprising the nucleic acid sequence of SEQ ID NO: 14.

19. A vector or plasmid comprising the nucleic acid molecule of claim 18.
